(19) United States
(12) Patent Application Publication
(10) Pub. No.: US 2010/0297082 A1
(43) Pub. Date: Nov. 25, 2010

(54) WEIGHT-BEARING POLYURETHANE COMPOSITES AND METHODS THEREOF

(75) Inventors: Scott A. Guelcher, Franklin, TN (US); Jerald E. Dumas, Nashville, TN (US); Todd M. Boyce, Matawan, NJ (US)

Correspondence Address: CHOATE, HALL & STEWART LLP, TWO INTERNATIONAL PLACE, BOSTON, MA, 02110, US

(73) Assignees: OSTEOTECH, INC., Eatontown, NJ (US); VANDERBILT UNIVERSITY, Nashville, TN (US)

(21) Appl. No.: 12/783,548

(22) Filed: May 19, 2010

Related U.S. Application Data
(60) Provisional application No. 61/179,621, filed on May 19, 2009.

(51) Int. Cl. A61F 2/28; B29C 43/02; A61P 19/00; A61K 48/00; A61K 35/12; A61K 38/18; A61K 38/02; A61K 38/43; A61K 31/195; A61K 47/34

(52) U.S. Cl. 424/93.1; 264/331.11; 424/93.2; 424/94.1; 514/7.6; 514/16.7; 514/44 R; 514/561; 514/772.3; 523/116

(57) ABSTRACT

Present inventions present composites of particles and polymers, as well as methods of making such composite and uses thereof. A low or non-porous composite comprises a plurality of bone particles; and polyurethane components with which the bone particles are combined. Provided composites can be prepared in a one-shot process. Alternatively or additionally, composites can be prepared by compression molding under a high pressure. Before or after implantation, provided composites may be set to form a hardened state of composites that provides mechanical strength and supports the in-growth of cells.

### CROSS REFERENCES OF RELATED APPLICATIONS

The present application claims priority under 35 U.S.C. §119(e) to U.S. provisional patent applications, U.S. Ser. No. 61/179,621, filed May 19, 2009; the entirety of which is incorporated herein by reference.

### GOVERNMENT SUPPORT

This invention was made with support from Center for Military Biomaterials Research (CeMBR) under Department of Defense (Grant No. USAM-RAA-W81XWH-04-2-0031). The U.S. government has certain rights to this invention.

### BACKGROUND

Bone is a composite material composed of impure hydroxyapatite, collagen, and a variety of non-collagenous proteins, as well as embedded and adherent cells. Bone can be processed into an implantable biomaterial, such as an allograft, for example, by removing the cells, leaving behind the extracellular matrix. The processed bone biomaterial can have a variety of properties, depending upon the specific processes and treatments applied to it, and may incorporate characteristics of other biomaterials with which it is combined. For example, bone-derived biomaterials may be processed into load-bearing mineralized grafts that support and integrate with the patient's own bone or may alternatively be processed into soft, moldable, or flowable demineralized bone biomaterials that have the ability to induce a cellular healing response.

The use of bone grafts and bone substitute materials in orthopedic medicine is well known. While bone wounds can regenerate without the formation of scar tissue, fractures and other orthopedic injuries take a long time to heal, during which the injured bone is unable to support physiologic loading. Metal pins, screws, and meshes are frequently needed to replace the mechanical functions of injured bone. However, metal is significantly stiffer than bone. Use of metal implants may result in decreased bone density around the implant site due to stress shielding. Furthermore, most metal implants are permanent and unable to participate in physiological remodeling.

Bone's cellular healing processes, through bone tissue formation by osteoblast cells coordinated with bone and graft resorption by osteoclast cells, permit bone grafts and certain bone substitute materials to remodel into endogenous bone that is almost indistinguishable from the original. However, the use of bone grafts is limited by the available shape and size of grafts and the desire to optimize both mechanical strength and degradation rate. Variations in bone size and shape among patients (and donors) also make bone grafts a less optimal substitute material. Bone substitute materials and bone chips are quickly remodeled but cannot immediately provide mechanical support, while cortical bone grafts can support physiological stresses but remodel slowly.

Thus, it is desirable to have a biomaterial for structural grafts that may be produced in larger quantities than grafts derived solely from bone and that may be fabricated or molded into shapes without being limited by the shape and/or mechanical strength of the originating tissue.

### SUMMARY

The invention relates to weight-bearing composites including at least a particulate component and a polymer component, methods of making such composites, methods of using such composites in orthopedic applications and various related compositions. In some embodiments, present invention provides low-porous or non-porous composites which, when implanted, providing osteoconductive pathway for cells into the interior of such implants, thus accelerating a remodeling process. Inventive composites comprise particles and polymers, such as a biocompatible polyurethane, and may further comprise additional components. In some embodiments, present invention provides compositions, methods and processes that can be used for the preparation of such composites. The invention also provides methods and kits for making and/or using such inventive low-porous or non-porous materials.

In some aspects, the present invention provides compositions and composites including a particulate component combined with a polymer component. A particle component may include, for example, a bone particle, bone-derived material, an inorganic material, a bone substitute material, a composite material, or any combinations thereof. A polymer component may include, for example, a monomer, a pre-polymer (e.g., an oligomer, a cross-linked polymer, a partially polymerized polymer, a partially cross-linked polymer, etc.), a polymer, or any combinations thereof.

In one aspect, the invention features a composite including allograft bone and biodegradable polyurethane (PUR). In some embodiments, a provided composite has a porosity less than 10%.

In some embodiments, provided composites are formed by combining a polyol, a polyisocyanate, and particles by compression molding.

In some embodiments, provided composites are formed by a “one-shot” process.

In general, composites provided herein are prepared for implantation. In some embodiments, a composite is prepared in a first state, that is flowable or moldable, and then is cured to a second, hardened state. In some embodiments, part or all of curing occurs after implantation. In some embodiments, a composite is compression molded and hardened prior to implantation. In some embodiments, a composite is prepared in a one-shot process and hardened before and/or after implantation. In some embodiments, a composite is workable so that it can be molded into an implantation site. Once cured, a composite may be a low-porous or non-porous composite including particles and polymers.

In some embodiments, a composition prepared and/or used in accordance with the present invention may include bone particles and a reactive liquid. Such a reactive liquid can be a two-component composition for generating a polyurethane. For example, such a reactive liquid may include a polyisocyanate (e.g., lysine diisocyanates (LDI), lysine triisocyanates (LTI), etc.) and a polyol that react to generate a polyurethane, and optionally may include one or more additional components such as a catalyst, a porogen, a chain extender, etc. In some embodiments, a composition may include one or more bioactive agents to be delivered such as one or more antibiotics, growth factors, etc.

In some aspects, the present invention features methods including contacting particles with polymer components to form composites. More specifically, in some embodiments, particles are combined with polymer components (e.g., a monomer, or a precursor, etc.) in a one-shot process. Composites may be compression molded under a high pressure.

In some embodiments, provided composites may be prepared using defatted, demineralized, or surface-demineralized particles (e.g., bone particles). In some embodiments, provided composites include polyurethane and/or polyurethane components. In some embodiments, provided composites are partially or completely hardened before implantation. In some embodiments, some hardening occurs after implantation. In some particular embodiments, compression molded composites are hardened prior to implantation.

In some embodiments, hardened composites are weight-bearing and have low or non-porosity.

Embodiments may include one or more of the following features or advantages. Composites can allow and encourage direct boney in-growth and remodeling, which can improve patient outcome. Composites can be formed into a variety of shapes and sizes. Methods of making composites can avoid additional processing steps. Methods of making can result in high mechanical properties of provided composites. Methods of making can enable longer storage stability of provided composites.

Other aspects, features and advantages will be apparent from the description of the following embodiments and from the claims.

### DEFINITIONS

The term “bioactive agent” is used herein to refer to compounds or entities that alter, promote, speed, prolong, inhibit, activate, or otherwise affect biological or chemical events in a subject (e.g., a human). For example, bioactive agents may include, but are not limited to osteogenic, osteoinductive, and osteoconductive agents, anti-HIV substances, anti-cancer substances, antibiotics, immunosuppressants, anti-viral agents, enzyme inhibitors, neurotoxins, opioids, hypnotics, anti-histamines, lubricants, tranquilizers, anti-convulsants, muscle relaxants, anti-Parkinson agents, anti-spasmodics and muscle contractants including channel blockers, miotics and anti-cholinergics, anti-glaucoma compounds, anti-parasite agents, anti-protozoal agents, and/or anti-fungal agents, modulators of cell-extracellular matrix interactions including cell growth inhibitors and anti-adhesion molecules, vasodilating agents, inhibitors of DNA, RNA, or protein synthesis, anti-hypertensives, analgesics, anti-pyretics, steroidal and non-steroidal anti-inflammatory agents, anti-angiogenic factors, angiogenic factors, anti-secretory factors, anticoagulants and/or antithrombotic agents, local anesthetics, ophthalmics, prostaglandins, anti-depressants, anti-psychotics, targeting agents, chemotactic factors, receptors, neurotransmitters, proteins, cell response modifiers, cells, peptides, polynucleotides, viruses, and vaccines. In certain embodiments, the bioactive agent is a drug. In certain embodiments, the bioactive agent is a small molecule.

A more complete listing of bioactive agents and specific drugs suitable for use in the present invention may be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, Edited by Susan Budavari et al., CRC Press, 1996, the United States Pharmacopeia-25/National Formulary-20, published by the United States Pharmcopeial Convention, Inc., Rockville Md., 2001, and the “Pharmazeutische Wirkstoffe”, edited by Von Keemann et al., Stuttgart/New York, 1987, all of which are incorporated herein by reference. Drugs for human use listed by the U.S. Food and Drug Administration (FDA) under 21 C.F.R. §§330.5, 331 through 361, and 440 through 460, and drugs for veterinary use listed by the FDA under 21 C.F.R. §§500 through 589, all of which are incorporated herein by reference, are also considered acceptable for use in accordance with the present invention.

The terms, “biodegradable”, “bioerodable”, or “resorbable” materials, as used herein, are intended to describe materials that degrade under physiological conditions to form a product that can be metabolized or excreted without damage to the subject. In certain embodiments, the product is metabolized or excreted without permanent damage to the subject. Biodegradable materials may be hydrolytically degradable, may require cellular and/or enzymatic action to fully degrade, or both. Biodegradable materials also include materials that are broken down within cells. Degradation may occur by hydrolysis, oxidation, enzymatic processes, phagocytosis, or other processes.

The term “biocompatible” as used herein, is intended to describe materials that, upon administration in vivo, do not induce undesirable side effects. In some embodiments, the material does not induce irreversible, undesirable side effects. In certain embodiments, a material is biocompatible if it does not induce long term undesirable side effects. In certain embodiments, the risks and benefits of administering a material are weighed in order to determine whether a material is sufficiently biocompatible to be administered to a subject.

The term “biomolecules” as used herein, refers to classes of molecules (e.g., proteins, amino acids, peptides, polynucleotides, nucleotides, carbohydrates, sugars, lipids, nucleoproteins, glycoproteins, lipoproteins, steroids, natural products, etc.) that are commonly found or produced in cells, whether the molecules themselves are naturally-occurring or artificially created (e.g., by synthetic or recombinant methods). For example, biomolecules include, but are not limited to, enzymes, receptors, glycosaminoglycans, neurotransmitters, hormones, cytokines, cell response modifiers such as growth factors and chemotactic factors, antibodies, vaccines, haptens, toxins, interferons, ribozymes, anti-sense agents, plasmids, DNA, and RNA. Exemplary growth factors include but are not limited to bone morphogenic proteins (BMP's) and their active fragments or subunits. In some embodiments, the biomolecule is a growth factor, chemotactic factor, cytokine, extracellular matrix molecule, or a fragment or derivative thereof, for example, a cell attachment sequence such as a peptide containing the sequence, RGD.

The term “carbohydrate” as used herein, refers to a sugar or polymer of sugars. The terms “saccharide”, “polysaccharide”, “carbohydrate”, and “oligosaccharide”, may be used interchangeably. Most carbohydrates are aldehydes or ketones with many hydroxyl groups, usually one on each carbon atom of the molecule. Carbohydrates generally have the molecular formula CₙH₂Oₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2:—fluororibose, deoxyribose, and hexose). Carbohydrates may exist in many different forms, for example, conformers, cyclic forms, acyclic forms, stereoisomers, tautomers, anomers, and isomers.

The term “composite” as used herein, is used to refer to a unified combination of two or more distinct materials. The composite may be homogeneous or heterogeneous. For example, a composite may be a combination of bone particles and a polymer; or a combination of bone particles, polymers and antibiotics. In certain embodiments, the composite has a particular orientation.

The term “demineralized” is used herein to refer to bone (e.g., particles) that have been subjected to a process that causes a decrease in the original mineral content. As utilized herein, the phrase “superficially demineralized” as applied to bone particles refers to bone particles possessing at least about 90% by weight of their original inorganic mineral content. The phrase “partially demineralized” as applied to the bone particles refers to bone particles possessing from about 8% to about 90% by weight of their original inorganic mineral content, and the phrase “fully demineralized” as applied to the bone particles refers to bone particles possessing less than about 8% by weight, for example, less than about 1% by weight, of their original inorganic mineral content. The unmodified term “demineralized” as applied to the bone particles is intended to cover any one or combination of the foregoing types of demineralized bone particles.

The term “deorganified” as herein applied to matrices, particles, etc., refers to bone or cartilage matrices, particles, etc., that were subjected to a process that removes at least part of their original organic content. In some embodiments, at least 1%, 10%, 20%, 30%, 40%, 50%, 60%, 70%, 80%, 90%, or 99% of the organic content of the starting material is removed. Deorganified bone from which substantially all the organic components have been removed is termed “anorganic.”

The term “flowable” as used herein, refers to a state of a composite/composition being able to flow. In some embodiments, flowability refers to an ability of a liquid to pass through a syringe (e.g, 8, 12 or 16 gauge syringes), or through a trocar. In some embodiments, composites used in the present application has less than 35 vol % particles (e.g., bone particles) and are flowable through a syring. In some embodiments, provided composites has less then 60 vol % particles (e.g., bone particles) and are flowable through a trocar. In some embodiments, provided composites has less than 70 vol % of particles, over which such composites may transit form a cohesive paste to a material that is not useful in accordance with the present invention.

The term “flowable or moldable polymer material” as used herein, refers to a flowable or moldable composition including one or more of monomers, pre-polymers (e.g., oligomers, low molecular weight polymers, uncross-linked polymers, partially cross-linked polymers, partially polymerized polymers), polymers, or combinations thereof that have been rendered flowable or moldable. One skilled in the art will recognize that a flowable or moldable polymer material need not be a polymer but may be polymerizable. In some embodiments, flowable or moldable polymer materials include polymers that have been heated past their glass transition or melting point. Alternatively or in addition, a flowable or moldable polymer material may include partially polymerized polymer, telechelic polymer, or other prepolymer. Alternatively or in addition, a flowable or moldable polymer material may be a polymer material/solvent mixture that sets when the solvent is removed. In some embodiments, a flowable or moldable polymer component/material is still flowable when adding particles and/or additional components.

The term “mineralized” as used herein, refers to bone that has been subjected to a process that caused a decrease in their original organic content (e.g., de-fatting, de-greasing). Such a process can result in an increase in the relative inorganic mineral content of the bone. Mineralization may also refer to the mineralization of a matrix such as extracellular matrix or demineralized bone matrix. The mineralization process may take place either in vivo or in vitro.

The term “non-demineralized” as herein applied to bone or bone particles, refers to bone or bone-derived material (e.g., particles) that have not been subjected to a demineralization process (i.e., a procedure that totally or partially removes the original inorganic content of bone).

The term “nontoxic” is used herein to refer to substances which, upon ingestion, inhalation, or absorption through the skin by a human or animal, do not cause, either acutely or chronically, damage to living tissue, impairment of the central nervous system, severe illness or death.

The term “osteoconductive” as used herein, refers to the ability of a substance or material to provide surfaces which are receptive to the growth of new bone.

The term “osteogenic” as used herein, refers to the ability of a substance or material that can induce bone formation.

The term “osteoinductive” as used herein, refers to the quality of being able to recruit cells (e.g., osteoblasts) from the host that have the potential to stimulate new bone formation. In general, osteoinductive materials are capable of inducing heterotopic ossification, that is, bone formation in extraskeletal soft tissues (e.g., muscle).

The term “osteoimplant” is used herein in its broadest sense and is not intended to be limited to any particular shapes, sizes, configurations, compositions, or applications. Osteoimplant refers to any device or material for implantation that aids or augments bone formation or healing. Osteoimplants are often applied at a bone defect site, e.g., one resulting from injury, defect brought about during the course of surgery, infection, malignancy, inflammation, or developmental malformation. Osteoimplants can be used in a variety of orthopedic, neurosurgical, dental, and oral and maxillofacial surgical procedures such as the repair of simple and compound fractures and non-unions, external, and internal fixations, joint reconstructions such as arthrodesis, general arthroplasty, deficit filling, disectomy, laminectomy, anterior cerival and thoracic operations, spinal fusions, etc.

The terms “polynucleotide”, “nucleic acid”, or “oligonucleotide” as used herein, refer to a polymer of nucleotides. The terms “polynucleotide”, “nucleic acid”, and “oligonucleotide”, may be used interchangeably. Typically, a polynucleotide comprises at least three nucleotides. DNAs and RNAs are exemplary polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thithymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyriboses, arabinose, and hexose), or modified phosphate groups (e.g., phosphorothioates and 5′-N-phosphoramidite linkages). The polymer may also be a short strand of nucleic acids such as RNAi, siRNA, or shRNA.

The terms “polypeptide”, “peptide”, or “protein” as used herein, include a string of at least three amino acids linked together by peptide bonds. The terms “polypeptide”, “peptide”, and “protein”, may be used interchangeably. In some embodiments, peptides may contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a peptide may be modified, for example, by the addition of a chemical entity such as a carbohydrate group, a phosphate group, a farnesyl group, an isofarnesyl group, a fatty acid group, a linker for conjugation, functionalization, or other modification, etc. In one embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

The terms “polysaccharide” or “oligosaccharide” as used herein, refer to any polymer or oligomer of carbohydrate residues. Polymers or oligomers may consist of anywhere from two to hundreds to thousands of sugar units or more. “Oligosaccharide” generally refers to a relatively low molecular weight polymer, while “polysaccharide” typically refers to a higher molecular weight polymer. Polysaccharides may be purified from natural sources such as plants or may be synthesized de novo in the laboratory. Polysaccharides isolated from natural sources may be modified chemically to change their chemical or physical properties (e.g., reduced, oxidized, phosphorylated, cross-linked). Carbohydrate polymers or oligomers may include natural sugars (e.g., glucose, fructose, galactose, mannose, arabinose, ribose, xylose, etc.) and/or modified sugars (e.g., 2′-fluororibose, 2′-deoxyribose, etc.). Polysaccharides may also be either straight or branched. They may contain both natural and/or unnatural carbohydrate residues. The linkage between the residues may be the typical ether linkage found in nature or may be a linkage only available to synthetic chemists. Examples of polysaccharides include cellulose, maltin, maltose, starch, modified starch, dextran, poly(dextrose), and fructose. In some embodiments, glycosaminoglycans are considered polysaccharides. Sugar alcohol, as used herein, refers to any polyol such as sorbitol, mannitol, xylitol, galactitol, erythritol, inositol, ribitol, dulcitol, adonitol, arabitol, dithioerythritol, dithiothreitol, glycerol, isomalt, and hydrogenated starch hydrolysates.

The term “porosity” as used herein, refers to the average amount of non-solid space contained in a material (e.g., a composite of the present invention). Such space is considered void of volume even if it contains a substance that is liquid at ambient or physiological temperature, e.g., 0.5° C. to 50° C. Porosity or void volume of a composite can be defined as the ratio of the total volume of the pores (i.e., void volume) in the material to the overall volume of composites. In some embodiments, porosity (ε), defined as the volume fraction pores, can be calculated from composite foam density, which can be measured gravimetrically. It will be appreciated by these of ordinary skill in the art that porosity of a provided composite may change over time, in some embodiments, after molding, setting or implantation. In some embodiments, particles (e.g., bone particles) function as a biologically active “porogen”, wherein pores are created as the allograft particles are resorbed. For the purpose of the present disclosure, porosity may be considered after molding and/or curing (i.e., setting). In some embodiments, the present invention provides composites having a porosity of less than about 10%. In some embodiments, provided composites have a porosity of less than about 10%, about 8%, about 6%, about 4%, about 2%, even nearly 0% or between any porosity of above after curing.

The term “prepolymer” as used herein, generally refers to any oligomers or other macromolecules that are capable of further polymerization. A prepolymer may be a low molecular weight oligomer typically produced through step growth polymerization. A pre-polymer may be formed with an excess of one of components to produce molecules that are all terminated with the same group. For example, a diol and an excess of a diisocyanate may be polymerized to produce isocyanate terminated prepolymer that may be combined with a diol to form a polyurethane. In some embodiments, polyisocyanate (e.g., LDI, LTI, etc.) is a prepolymer. In some embodiments, polyisocyanate is reacted with polyols to form quasi-prepolymers. For example, WO 2007123536 provides exemplary prepolymers and methods of using them, the content of which are incorporated by reference herein. In certain embodiments, such quasi-prepolymers are not used in accordance with the present invention.

The term “remodeling” as used herein, describes the process by which native bone, processed bone allograft, whole bone sections employed as grafts, and/or other bony tissues are replaced with new cell-containing host bone tissue by the action of osteoclasts and osteoblasts. Remodeling also describes the process by which non-bony native tissue and tissue grafts are removed and replaced with new, cell-containing tissue in vivo. Remodeling also describes how inorganic materials (e.g., calcium-phosphate materials, such as β-tricalcium phosphate) is replaced with living bone.

The term “setting time” as used herein, is approximated by the tack-free time (TFT), which is defined as the time at which the material could be touched with a spatula with no adhesion of the spatula to the foam. At the TFT, the wound could be closed without altering the properties of the material. The terms “set”, “cure” and “harden” as used herein may be interchangeable.

The term “shaped” as used herein, is intended to characterize a material (e.g., composite) or an osteoimplant refers to a material or osteoimplant of a determined or regular form or configuration in contrast to an indeterminate or vague form or configuration (as in the case of a lump or other solid matrix of special form). Materials may be shaped into any shape, configuration, or size. For example, materials can be shaped as sheets, blocks, plates, disks, cones, pins, screws, tubes, teeth, bones, portions of bones, wedges, cylinders, threaded cylinders, and the like, as well as more complex geometric configurations.

The term “small molecule” as used herein, is used to refer to molecules, whether naturally-occurring or artificially created (e.g., via chemical synthesis), that have a relatively low molecular weight. In some embodiments, small molecules have a molecular weight of less than about 2,500 g/mol, for example, less than 1000 g/mol. In certain embodiments, small molecules are biologically active in that they produce a local or systemic effect in animals, such as mammals, e.g., humans. In certain embodiments, a small molecule is a drug. In certain embodiments, though not necessarily, a drug is one that has already been deemed safe and effective for use by an appropriate governmental agency or body (e.g., the U.S. Food and Drug Administration).

The term “transformation” as used herein, describes a process by which a material is removed from an implant site and replaced by host tissue after implantation. Transformation may be accomplished by a combination of processes, including but not limited to remodeling, degradation, resorption, and tissue growth and/or formation. Removal of the material may be cell-mediated or accomplished through chemical processes, such as dissolution and hydrolysis.

The term “wet compressive strength” as used herein, refers to the compressive strength of an osteoimplant after being immersed in physiological saline (e.g., phosphate-buffered saline (PBS), water containing 0.9 g NaCl/100 ml water, etc.) for a minimum of 12 hours (e.g., 24 hours). Compressive strength and modulus are well-known measurements of mechanical properties and is measured using the procedure described herein.

The term “working time” as used herein, is defined in the ISO9917 standard as “the period of time, measured from the start of mixing, during which it is possible to manipulate a dental material without an adverse effect on its properties” (Clarkin et al., J Mater Sci: Mater Med 2009; 20:1563-1570). In some embodiments, the working time for a two-component polyurethane is determined by the gel point, the time at which the crosslink density of the polymer network is sufficiently high that the material gels and no longer flows.

### DESCRIPTION OF DRAWING

FIG. 1 depicts characterization of rabbit mineralized bone particles. Low magnification SEM images of (A) MBP and (B) SDBP showing negligible changes in size and shape after surface demineralization. High magnification SEM images of (C) MBP and (D) SDBP particles showing the exposure of collagen fibrils on the surface after demineralization, (E) composition of the surface of MBP and SDBP measured by XPS, and (F) particle size distribution measured by laser diffraction (Micromeritics).

FIG. 2 depicts results from a fluorescein isothiocyanate (FITC) assay illustrating that surface-demineralization enhances the reactivity of rabbit allograft bone particles. Rabbit MBP and SDBP were incubated in a FITC solution (7 mg/ml) in 1 ml borate buffer for 1 hour. As a negative FITC-untreated control, only borate buffer was added to three of the MBP samples. After washing with borate buffer solution, the MBP and SDBP were suspended in 0.1 mL borate buffer and transferred to a 96 well plate. As a positive FITC-treated control, the tissue culture polystyrene well plate was also incubated in FITC solution. MBP in borate buffer was used as a control in this study. The fluorescence of each well was read using a FL600 microplate fluorescence reader at an absorption of 495 nm and an emission at 525 nm.

FIG. 3 depicts IR Spectra of 6C3G1L600-SDBP composite (blue) and mineralized bone particles (red). The absence of a peak at 2285-2250 cm⁻¹, marked by the black arrow, indicates that there is a negligible amount of free NCO. Most peaks are overlapping between the MBP/PUR composite and the MBP with the exception of the ester and urethane carbonyl peaks.

FIG. 4 depicts allograft particles are more uniformly distributed in 300 MW composites compared to 600 MW SDBP composites. A: 6C3G1L300-MBP core, B: 6C3G1L600-SDBP core, C: region of interest, D: Volume fractions of bone and polymer measured by histomorphometry (n=3) show higher variability in the center region of the implant for 600 MW SDBP compared to the other treatment groups with a significant difference between the 6C3G1L300-MBP and 6C3G1L600-SDBP groups.

FIG. 5 depicts radiographs of extracted rabbit distal femurs. A: 6C3G1L300-MBP, B: 6C3G1L300-SDBP, C: 6C3G1L600-MBP, D: 6C3G1L600-SDBP.

FIG. 6 depicts histology at 2 weeks for 6C3G1L300-SDBP treatment group. (A)-(D) Histological sections of the 6C3G1L300-SDBP treatment group are stained with Sanderson's rapid bone stain. (A) At two weeks, there is evidence of bone apposition and the composite is encapsulated in a bony shell (1.25×). (B)-(D) Higher magnification images (10×-20×) show bone apposition (yellow asterisk), resorption (black asterisk) and remodeling of the allograft component (A) via the process of creeping substitution, and polymer (P) degradation (20×).

FIG. 7 depicts low magnification (1.25×) histological sections of all treatment groups at 6 weeks. A: 6C3G1L300-MBP, B: 6C3G1L300-SDBP, C: 6C3G1L600-MBP, D: 6C3G1L600-SDBP.

FIG. 8 depicts remodeling of allograft bone particles in 6C3G1L600-SDBP treatment group. (A)-(B): Cellular infiltration (CI) and new bone formation (NB) around the edge of SDBP. Osteocytes are stained blue within the new bone matrix. (20×-40×), (A) shows the union of two allograft particles (labeled as A) by new bone (C): Both new bone formation and resorption (black asterisk) of SDBP (40×), (D): New bone ingrowth around an island of residual polymer (P) at the edge of the host bone, which is lined by osteoid. (10×).

FIG. 9 depicts allograft resorption, polymer degradation, cellular infiltration, and new bone formation in MBP composites incorporating PUR networks with 300 g/mol and 600 g/mol polyester triols. (A) Rectangular region of interest (1.8×3.9 mm). (B) Histomorphometric analysis of an active region of remodeling shows that composites fabricated from the 600 g/mol polyol exhibit faster polymer degradation, cellular infiltration, and new bone formation relative to those prepared from the 300 g/mol polyol, but the differences were not significant. Differences in allograft resorption were significant at p<0.06.

FIG. 10 depicts the process of creeping substitution is accelerated by the presence of a continuous, percolated bone phase. Remodeling of MBP/PUR composite occurring around the un-remodeled core. (A) 10× micrograph near the boundary between an actively remodeling region and the un-remodeled core. (B) An area of active of active remodeling outside the un-remodeled core (20×). (C) A region enriched in polymer where the residual polymer hinders the penetration of cells (yellow asterisk) and a region where bone particle contacts provide a pathway for infiltration (pink asterisk) (20×).

FIG. 11 depicts representative low magnification (4×) view shows extensive cellular infiltration (light blue), polymer degradation, and new bone formation in a 6C3G1L300-MBP implant. (B)-(C) Higher magnification (10×-20×) of host bone ingrowth lined with osteoid (yellow asterisk). (D) High magnification (20×) of new bone approximately 2 mm deeper into the implant cavity.

FIG. 12 shows compressive strength of composites made from (a) LDI quasi-prepolymer (QP) and by (b) a one-shot process.

FIG. 13 shows LTI one-shot approach shits optimal index (on the x-axis).

FIG. 14 shows effect of splitting the bone between the LTI or polyol.

FIG. 15 shows dynamic mechanical analysis data, where storage modulus is plotted as a function of frequency.

### DETAILED DESCRIPTION OF CERTAIN EMBODIMENTS

As used herein and in the appended claims, the singular forms “a,” “an” and “the” include plural references unless the content clearly dictates otherwise. All publications, patent applications, patents, and other references mentioned herein are incorporated by reference in their entirety.

Bone/polyurethane composites described herein include bone (e.g., bone particles), polyurethane, and in some embodiments, one or more additional components (e.g., a porogen and/or a bioactive agent). As described below, bone and biodegradable polyurethanes are combined to form a porous composite (e.g., an osteoimplant). In some embodiments, porous composites retain strength and/or release bioactive agents when present in a body. In some embodiments, composites degrade and are replaced by new tissue.

Inventive composites can be used in a large variety of clinical applications, for example, as bone void fillers, to repair or help healing of skeletal deficiencies resulting from trauma, tumors, surgery, iatrogenic, congenital, genetic, metabolic and degenerative or abnormal development, and inflammatory infection. In some embodiments, inventive composites promote cellular infiltration from adjacent osseous tissues, thus accelerating the remodeling process.

The invention also provides methods of preparing and using inventive composites as well as kits for preparing and/or administering inventive composites. Inventive porous composites may be prepared using any of a variety of methods. In some embodiments, inventive composites are prepared using a method that includes water as a blowing agent. In one embodiment, bone particles or other bone substitute materials are combined with polyurethanes and injected, extruded, molded, or similarly delivered to a tissue site (e.g., bony defect) of a subject. Inventive composites are engineered to set in situ to form a solid composite that may have a desired or predetermined mechanical strength. In certain embodiments, polyurethane present in a composition or composite may include monomers or pre-polymers. In some embodiments, polyurethane is a polymer that has been rendered formable through combination of two liquid components (i.e., a polyisocyanate prepolymer and a polyol).

Particulate Component

Particles used in accordance with the present invention may include a bone-derived material, an inorganic material, a bone substitute material, a composite material, or any combinations thereof.

Bone Particles. Any kind of bone and/or bone-derived particles may be used in the present invention. In some embodiments, bone particles employed in the preparation of bone particle-containing composites are obtained from cortical, cancellous, and/or corticocancellous bone. Bone particles may be obtained from any vertebrate. Bone may be of autogenous, allogenic, and/or xenogeneic origin. In certain embodiments, bone particles are autogenous, that is, bone particles are from the subject being treated. In other embodiments, bone particles are allogenic (e.g., from donors). In certain embodiments, the source of bone may be matched to the eventual recipient of inventive composites (i.e., the donor and recipient are of the same species). For example, human bone particle is typically used in a human subject. In certain embodiments, bone particles are obtained from cortical bone of allogenic origin. In certain embodiments, bone particles are obtained from bone of xenogeneic origin. Porcine and bovine bone are types of xenogeneic bone tissue that can be used individually or in combination as sources for bone particles and may offer advantageous properties. Xenogenic bone tissue may be combined with allogenic or autogenous bone.

Bone particles can be formed by any process known to break down bone into small pieces. Exemplary processes for forming such particles include milling whole bone to produce fibers, chipping whole bone, cutting whole bone, grinding whole bone, fracturing whole bone in liquid nitrogen, or otherwise disintegrating the bone. Bone particles can optionally be sieved to produce particles of a specific size range. Bone particles may be of any shape or size. Exemplary shapes include spheroidal, plates, shards, fibers, cuboidal, sheets, rods, oval, strings, elongated particles, wedges, discs, rectangular, polyhedral, etc.

In some embodiments, bone particles have a medium or mean diameter about 1200 microns, 1100 microns, 1000 microns, 900 microns, 800 microns, 700 microns, 600 microns, 500 microns, 400 microns, 300 microns, 200 microns, 100 microns, etc. In some embodiments, diameters of bone particles are within a range between any of such sizes. For example, medium or mean diameters of bone particles have a range from approximately 100 microns to approximately 1000 microns.

As for irregularly shaped bone particles, recited dimension ranges may represent the length of the greatest or smallest dimension of the particle. As examples, bone particles can be pin shaped, with tapered ends having an average diameter of from about 100 microns to about 500 microns. As will be appreciated by one of skill in the art, for injectable composites, the maximum particle size will depend in part on the size of the cannula or needle through which the material will be delivered.

In some embodiments, particle size distribution of bone particles utilized in accordance with the present inventions with respect to a mean value or a median value may be plus or minus, e.g., about 10% or less of the mean value, about 20% or less of the mean value, about 30% or less of the mean value, about 40% or less of the mean value, about 50% or less of the mean value, about 60% or less of the mean value, about 70% or less of the mean value, about 80% or less of the mean value, or about 90% or less of the mean value.

In some embodiments, bone particles have a median or mean length of about 1200 microns, 1100 microns, 1000 microns, 900 microns, 800 microns, 700 microns, 600 microns, 500 microns, 400 microns, 300 microns, 200 microns, 100 microns, etc. In some embodiments, about 70, about 80 or about 90 percent of bone particles possess a median or mean length within a range of any of such sizes.

For bone particles that are fibers or other elongated particles, in some embodiments, at least about 90 percent of the particles possess a median or mean length in their greatest dimension in a range from approximately 100 microns to approximately 1000 microns. Particles may possess a median or mean length to median or mean thickness ratio from at least about 5:1 up to about 500:1, for example, from at least about 50:1 up to about 500:1, or from about 50:1 up to about 100:1; and a median or mean length to median or mean width ratio of from about 10:1 to about 200:1 and, for example, from about 50:1 to about 100:1. In certain embodiments, bone particles are short fibers having a cross-section of about 300 microns to about 100 microns and a length of about 0.1 mm to about 1 mm.

Processing of bone to provide particles may be adjusted to optimize for the desired size and/or distribution of bone particles. The properties of resulting inventive composites (e.g., mechanical properties) may also be engineered by adjusting weight percent, shapes, sizes, distribution, etc. of bone particles or other particles. For example, an inventive composite may be made more viscous and load bearing by including a higher percentage of particles.

U.S. Pat. Nos. 5,899,939; 5,507,813; 6,123,731; 6,294,041; 6,294,187; 6,332,779; 6,440,444; and 6,478,825; the contents of all of which are incorporated herein by reference, describe methods for preparing composites including allogenic bone for use in orthopedic applications.

Bone particles utilized in accordance with the present inventions may be demineralized, non-demineralized, mineralized, defatted and/or anorganic. In some embodiments, bone particles are used “as is” in preparing inventive composites. In some embodiments, bone particles are defatted and/or disinfected. An exemplary defatting/disinfectant solution is an aqueous solution of ethanol. Other organic solvent may also be used in the defatting and disinfecting bone particles. For example, methanol, isopropanol, butanol, DMF, DMSO, diethyl ether, hexanes, glyme, tetrahydrofuran, chloroform, methylene chloride, and carbon tetrachloride may be used. In certain embodiments, a non-halogenated solvent is used. A defatting/disinfecant solution may also include a detergent (e.g., an aqueous solution of a detergent). Ordinarily, at least about 10 to about 40 percent by weight of water (i.e., about 60 to about 90 weight percent of defatting agent such as alcohol) should be present in the defatting/disinfecting solution to produce optimal lipid removal and disinfection within the shortest period of time. An exemplary concentration range of a defatting solution is from about 60 to about 85 weight percent alcohol, for example, about 70 weight percent alcohol.

In some embodiments, bone particles are demineralized. Bone particles can be optionally demineralized in accordance with known and/or conventional procedures in order to reduce their inorganic mineral content. Demineralization methods remove the inorganic mineral component of bone by employing acid solutions. Such methods are well known in the art, see for example, Reddi, et al., Proc. Nat. Acad. Sci., 1972, 69:1601-1605, the contents of which are incorporated herein by reference. The strength of the acid solution, the shape and dimensions of the bone particles and the duration of the demineralization treatment will determine the extent of demineralization. Reference in this regard is made to Lewandrowski, et al., J. Biomed. Mater. Res., 1996, 31:365-372 and U.S. Pat. No. 5,290,558, the contents of both of which are incorporated herein by reference.

In an exemplary defatting/disinfecting/demineralization procedure, bone particles are subjected to a defatting/disinfecting step, followed by an acid demineralization step. An exemplary defatting/disinfectant solution is an aqueous solution of ethanol. In some embodiments, at least about 10 to about 40 percent by weight of water (i.e., about 60 to about 90 weight percent of defatting agent such as alcohol) can be present in a defatting/disinfecting solution to produce optimal lipid removal and disinfection within a reasonable period of time. An exemplary concentration range of a defatting solution is from about 60 to about 85 weight percent alcohol, for example, about 70 weight percent alcohol. Ethanol is typically the alcohol used in this step; however, other alcohols such as methanol, propanol, isopropanol, denatured ethanol, etc. may also be used. Following defatting, bone particles can be immersed in acid over time to effect their demineralization. The acid also disinfects the bone by killing viruses, vegetative microorganisms, and spores. Acids which can be employed in this step include inorganic acids such as hydrochloric acid and organic acids such as peracetic acid. After acid treatment, demineralized bone particles can be rinsed with sterile water to remove residual amounts of acid and thereby raise the pH. Bone particles may be dried, for example, by lyophilization, before being incorporated into a composite. Bone particles may be stored under aseptic conditions, for example, in a lyophilized state, until they are used or sterilized using known methods (e.g., gamma irradiation) shortly before combining them with polyurethanes used in inventive composites.

As utilized herein, the phrase “superficially demineralized” as applied to the bone particles refers to bone particles possessing at least about 90% by weight of their original inorganic mineral content. The phrase “partially demineralized” as applied to the bone particles refers to bone particles possessing from about 8% to about 90% weight of their original inorganic mineral content, and the phrase “fully demineralized” as applied to the bone particles refers to bone particles possessing less than about 8%, preferably less than about 1%, by weight of their original inorganic mineral content. The unmodified term “demineralized” as applied to the bone particles is intended to cover any one or combination of the foregoing types of demineralized bone particles, that is, superficially demineralized, partially demineralized, or fully demineralized bone particles.

In alternative embodiments, surfaces of bone particles may be lightly demineralized according to the procedures in our commonly owned U.S. patent application Ser. No. 10/285,715, filed Nov. 1, 2002, published as U.S. Patent Publication No. 2003/0144743, on Jul. 31, 2003, the contents of which are incorporated herein by reference. Even minimal demineralization, for example, of less than 5% removal of the inorganic phase, increases the hydroxylation of bone fibers and the surface concentration of amine groups. Demineralization may be so minimal, for example, less than 1%, that the removal of the calcium phosphate phase is almost undetectable. Rather, the enhanced surface concentration of reactive groups defines the extent of demineralization. This may be measured, for example, by titrating the reactive groups. Surface composition can also be measured by x-ray photoelectron spectroscopy (XPS), an experimental technique that measures the atomic composition of the top 1-10 nm of the surface. In some embodiments, in a polymerization reaction that utilizes the exposed allograft surfaces to initiate a reaction, the amount of unreacted monomer remaining may be used to estimate reactivity of the surfaces. Surface reactivity may be assessed by a surrogate mechanical test, such as a peel test of a treated coupon of bone adhering to a polymer.

In certain embodiments, bone particles are subjected to a process that partially or totally removes their initial organic content to yield mineralized and anorganic bone particles, respectively. Different mineralization methods have been developed and are known in the are (Hurley, et al., Milit. Med. 1957, 101-104; Kershaw, Pharm. J. 6:537, 1963; and U.S. Pat. No. 4,882,149; each of which is incorporated herein by reference). For example, a mineralization procedure can include a de-greasing step followed by a basic treatment (with ammonia or another amine) to degrade residual proteins and a water washing (U.S. Pat. Nos. 5,417,975 and 5,573,771; both of which are incorporated herein by reference). Another example of a mineralization procedure includes a defatting step where bone particles are sonicated in 70% ethanol for 1-3 hours.

In some embodiments, bone particles can be modified in one or more ways, e.g., their protein content can be augmented or modified as described, for example, in U.S. Pat. Nos. 4,743,259 and 4,902,296, the contents of both of which are incorporated herein by reference.

Mixtures or combinations of one or more of the foregoing types of bone particles can be employed. For example, one or more of the foregoing types of demineralized bone particles can be employed in combination with non-demineralized bone particles, i.e., bone particles that have not been subjected to a demineralization process, or inorganic materials. The amount of each individual type of bone particle employed can vary widely depending on the mechanical and biological properties desired. Thus, in some embodiments, mixtures of bone particles of various shapes, sizes, and/or degrees of demineralization may be assembled based on the desired mechanical, thermal, chemical, and biological properties of a composite. A desired balance between the various properties of composites (e.g., a balance between mechanical and biological properties) may be achieved by using different combinations of particles. Suitable amounts of various particle types can be readily determined by those skilled in the art on a case-by-case basis by routine experimentation.

The differential in strength, osteogenicity, and other properties between partially and fully demineralized bone particles on the one hand, and non-demineralized, superficially demineralized bone particles, inorganic ceramics, and other bone substitutes on the other hand can be exploited. For example, in order to increase the compressive strength of an osteoimplant, the ratio of nondemineralized and/or superficially demineralized bone particles to partially or fully demineralized bone particles may favor the former, and vice versa. Bone particles in composites also play a biological role. Non-demineralized bone particles bring about new bone in-growth by osteoconduction. Demineralized bone particles likewise play a biological role in bringing about new bone in-growth by osteoinduction. Both types of bone particles are gradually remodeled and replaced by new host bone as degradation of the composite progresses over time. Thus, the use of various types of bone particles can be used to control the overall mechanical and biological properties, (e.g., strength, osteoconductivity, and/or osteoinductivity, etc.) of osteoimplants.

Surface Modification. Particles utilized in accordance with the present invention may be optionally treated to enhance their interaction with polymer components (e.g., polyurethanes) and/or to confer some properties to particle surface.

While some bone particles will interact readily with monomers and be covalently linked to polyurethane matrices, it may be desirable to modify surface of bone particles to facilitate their incorporation into polymers that do not bond well to bone, such as poly(lactides). Surface modification may provide a chemical substance that is strongly bonded to the surface of bone, e.g., covalently bonded to the surface. Bone particles may, alternatively or additionally, be coated with a material to facilitate interaction with polymers of inventive composites.

In some embodiments, silane coupling agents are employed to link a monomer or initiator molecule to the surface of bone particles. Silane has at least two sections, a set of leaving groups and at least an active group. An active group may be connected to the silicon atom in the silane by an elongated tether group. An exemplary silane coupling agent is 3-trimethoxysilylpropylmethacrylate, available from Union Carbide. Three methoxy groups are leaving groups, and the methacrylate active group is connected to the silicon atom by a propyl tether group. In some embodiments, a leaving group is an alkoxy group such as methoxy or ethoxy. Depending on the solvent used to link the coupling agent to bone particles, hydrogen or alkyl groups such as methyl or ethyl may serve as leaving groups. The length of tethers determines the intimacy of connection between polymers and bone particles. By providing a spacer between bone particles and active groups, the tether also reduces competition between chemical groups at the particle surface and the active group and makes the active group more accessible to monomers during polymerization.

In some embodiments, an active group is an analog of monomers of a polymer used in inventive composites. For example, amine active groups will be incorporated into polyurethane matrices, copolymers (e.g., polyesters, polycarbonates, polycaprolactone), and other polymer classes based on monomers that react with amines, even if the polymer does not contain an amine. Hydroxy-terminated silanes will be incorporated into polyamino acids, polyesters, polycaprolactone, polycarbonates, polyurethanes, and other polymer classes that include hydroxylated monomers. Aromatic active groups or active groups with double bonds will be incorporated into vinyl polymers and other polymers that grow by radical polymerization (e.g., polyacrylates, polymethacrylates). It is not necessary that the active group be monofunctional. Indeed, it may be preferable that active groups that are to be incorporated into polymers via step polymerization be difunctional. A silane having two amines, even if one is a secondary amine, will not terminate a polymer chain but can react with ends of two different polymer chains. Alternatively, the active group may be branched to provide two reactive groups in the primary position.

An exemplary list of silanes that may be used with the present invention is provided in U.S. Patent Publication No. 2004/0146543, the contents of which are incorporated herein by reference. Silanes are available from companies such as Union Carbide, AP Resources Co. (Seoul, South Korea), and BASF. Where a silane contains a potentially non-biocompatible moiety as the active group, it may be used to tether a biocompatible compound to bone particles using a reaction in which the non-biocompatible moiety is a leaving group. It may be desirable to attach the biocompatible compound to the silane before attaching the silane to the bone particle, regardless of whether the silane is biocompatible or not. The derivatized silanes may be mixed with silanes that can be incorporated directly into the polymer and reacted with bone particles, coating the bone particles with a mixture of “bioactive” silanes and “monomer” silanes. U.S. Pat. No. 6,399,693, the contents of which are incorporated herein by reference discloses composites of silane modified polyaromatic polymers and bone. In some embodiments, silane-derivatized polymers may be used in inventive composites instead of or in addition to first silanizing bone particles. In certain embodiments, polyurethanes and any copolymers used in accordance with the present inventions may not include silane modified polyaromatic polymers.

The active group of silanes may be incorporated directly into polymers or may be used to attach a second chemical group to bone particles. For example, if a particular monomer polymerizes through a functional group that is not commercially available as a silane, the monomer may be attached to the active group.

Non-silane linkers may also be employed to produce composites according to the invention. For example, isocyanates will form covalent bonds with hydroxyl groups on the surface of hydroxyapatite ceramics (de Wijn, et al., Fifth World Biomaterials Congress, May 29-Jun. 2, 1996, Toronto, Calif.). Isocyanate anchors, with tethers and active groups similar to those described with respect to silanes, may be used to attach monomer-analogs to bone particles or to attach chemical groups that will link covalently or non-covalently with a polymer side group. Polyamines, organic compounds containing one or more primary, secondary, or tertiary amines, will also bind with both the bone particle surface and many monomer and polymer side groups. Polyamines and isocyanates may be obtained from Aldrich.

Alternatively or additionally, biologically active compounds such as a biomolecule, a small molecule, or a bioactive agent may be attached to bone particles through a linker. For example, mercaptosilanes will react with sulfur atoms in proteins to attach them to bone particles. Aminated, hydroxylated, and carboxylated silanes will react with a wide variety functional groups. Of course, the linker may be optimized for the compound being attached to bone particles.

Biologically active molecules can modify non-mechanical properties of inventive composites as they degrade. For example, immobilization of a drug on bone particles allows it to be gradually released at an implant site as the composite degrades. Anti-inflammatory agents embedded within inventive composites will control inflammatory response long after an initial response to injection of the composites. For example, if a piece of the composite fractures several weeks after injection, immobilized compounds will reduce the intensity of any inflammatory response, and the composite will continue to degrade through hydrolytic or physiological processes. In some embodiments, compounds may also be immobilized on the bone particles that are designed to elicit a particular metabolic response or to attract cells to injection sites.

Some biomolecules, small molecules, and bioactive agents may also be incorporated into polyurethane matrices used in inventive composites. For example, many amino acids have reactive side chains. The phenol group on tyrosine has been exploited to form polycarbonates, polyarylates, and polyiminocarbonates (see Pulapura, et al., Biopolymers, 1992, 32: 411-417; and Hooper, et al., J. Bioactive and Compatible Polymers, 1995, 10:327-340, the entire contents of both of which are incorporated herein by reference). Amino acids such as lysine, arginine, hydroxylysine, proline, and hydroxyproline also have reactive groups and are essentially tri-functional. Amino acids such as valine, which has an isopropyl side chain, are still difunctional. Such amino acids may be attached to the silane and still leave one or two active groups available for incorporation into a polymer.

Non-biologically active materials may also be attached to bone particles. For example, radiopaque (e.g., barium sulfate), luminescent (e.g., quantum dots), or magnetically active particles (e.g., iron oxide) may be attached to bone particles using the techniques described above. Mineralized bone particles are an inherently radiopaque component of some embodiments of present inventions, whereas demineralized bone particles, another optional component of inventive composites, are not radiopaque. To enhance radiopacity of inventive composites, mineralized bone particles can be used. Another way to render radiopaque the polymers utilized in accordance with the present inventions, is to chemically modify them such that a halogen (e.g., iodine) is chemically incorporated into the polyurethane matrices, as in U.S. patent application Ser. No. 10/952,202, now published as U.S. Patent Publication No. 2006-0034769, whose content is incorporated herein by reference.

If a material, for example, a metal atom or cluster, cannot be produced as a silane or other group that reacts with bone particles, then a chelating agent may be immobilized on bone particle surface and allowed to form a chelate with the atom or cluster. As bone particles and polymers used in the present invention are resorbed, these non-biodegradable materials may be removed from tissue sites by natural metabolic processes, allowing degradation of the polymers and resorption of the bone particles to be tracked using standard medical diagnostic techniques.

In some embodiments, bone particles is surface-demineralized. In some embodiments, bone particle surface is chemically treated before being mixed with polyurethane. For example, non-demineralized bone particles may be rinsed with phosphoric acid, e.g., for 1 to 15 minutes in a 5-50% solution by volume. Those skilled in the art will recognize that the relative volume of bone particles and phosphoric acid solution (or any other solution used to treat bone particles), may be optimized depending on the desired level of surface treatment. Agitation will also increase the uniformity of the treatment both along individual particles and across an entire sample of particles. A phosphoric acid solution reacts with mineral components of bone particles to coat the bone particles with calcium phosphate, which may increase the affinity of the surface for inorganic coupling agents such as silanes and for polymer components of the composite. As noted above, bone particle surface may be partially demineralized to expose the collagen fibers.

Collagen fibers exposed by demineralization are typically relatively inert but have some exposed amino acid residues that can participate in reactions. Collagen may be rendered more reactive by fraying triple helical structures of the collagen to increase exposed surface area and number of exposed amino acid residues. This not only increases surface area of bone particles available for chemical reactions but also for their mechanical interactions with polymers as well. Rinsing partially demineralized bone particles in an alkaline solution will fray collagen fibrils. For example, bone particles may be suspended in water at a pH of about 10 for about 8 hours, after which the solution is neutralized. One skilled in the art will recognize that this time period may be increased or decreased to adjust the extent of fraying. Agitation, for example, in an ultrasonic bath, may reduce the processing time. Alternatively or additionally, bone particles may be sonicated with water, surfactant, alcohol, or some combination of these.

In some embodiments, collagen fibers at bone particle surface may be cross-linked. A variety of cross-linking techniques suitable for medical applications are well known in the art (see, for example, U.S. Pat. No. 6,123,781, the contents of which are incorporated herein by reference). For example, compounds like 1-ethyl-3-(3-dimethylaminopropyl) carbodiimide hydrochloride, either alone or in combination with N-hydroxysuccinimide (NHS) will crosslink collagen at physiologic or slightly acidic pH (e.g., in pH 5.4 MES buffer). Acyl azides and genipin, a naturally occurring bicyclic compound including both carboxylate and hydroxyl groups, may also be used to cross-link collagen chains (see Simmons, et al, Biotechnol. Appl. Biochem., 1993, 17:23-29; PCT Publication WO98/19718, the contents of both of which are incorporated herein by reference). Alternatively or additionally, hydroxymethyl phosphine groups on collagen may be reacted with the primary and secondary amines on neighboring chains (see U.S. Pat. No. 5,948,386, the entire contents of which are incorporated herein by reference). Standard cross-linking agents such as mono- and dialdehydes, polyepoxy compounds, tanning agents including polyvalent metallic oxides, organic tannins, and other plant derived phenolic oxides, chemicals for esterification or carboxyl groups followed by reaction with hydrazide to form activated acyl azide groups, dicyclohexyl carbodiimide and its derivatives and other heterobifunctional crosslinking agents, hexamethylene diisocyanate, and sugars may also be used to cross-link collagens. Bone particles are then washed to remove all leachable traces of materials. In other embodiments, enzymatic cross-linking agents may be used. Additional cross-linking methods include chemical reaction, irradiation, application of heat, dehydrothermal treatment, enzymatic treatment, etc. One skilled in the art will easily be able to determine the optimal concentrations of cross-linking agents and incubation times for the desired degree of cross-linking.

Both frayed and unfrayed collagen fibers may be derivatized with monomer, pre-polymer, oligomer, polymer, initiator, and/or biologically active or inactive compounds, including but not limited to biomolecules, bioactive agents, small molecules, inorganic materials, minerals, through reactive amino acids on the collagen fiber such as lysine, arginine, hydroxylysine, proline, and hydroxyproline. Monomers that link via step polymerization may react with these amino acids via the same reactions through which they polymerize. Vinyl monomers and other monomers that polymerize by chain polymerization may react with these amino acids via their reactive pendant groups, leaving the vinyl group free to polymerize. Alternatively, or in addition, bone particles may be treated to induce calcium phosphate deposition and crystal formation on exposed collagen fibers. Calcium ions may be chelated by chemical moieties of the collagen fibers, and/or calcium ions may bind to the surface of the collagen fibers. James et al., Biomaterials 20:2203-2313, 1999; incorporated herein by reference. The calcium ions bound to the collagen provides a biocompatible surface, which allows for the attachment of cells as well as crystal growth. The polymer will interact with these fibers, increasing interfacial area and improving the wet strength of the composite.

In some embodiments, the surface treatments described above or treatments such as etching may be used to increase the surface area or surface roughness of bone particles. Such treatments increase the interfacial strength of the particle/polymer interface by increasing the surface area of the interface and/or the mechanical interlocking of bone particles and polyurethane. Such surface treatments may also be employed to round the shape or smooth the edges of bone particles to facilitate delivery of the inventive composite. Such treatment is particularly useful for injectable composites.

In some embodiments, surface treatments of bone particles are optimized to enhance covalent attractions between bone particles and polyurethanes. In some embodiments, the surface treatment may be designed to enhance non-covalent interactions between bone particle and polyurethane matrix. Exemplary non-covalent interactions include electrostatic interactions, hydrogen bonding, pi-bond interactions, hydrophobic interactions, van der Waals interactions, and mechanical interlocking. For example, if a protein or a polysaccharide is immobilized on bone particle, the chains of polymer matrix will become physically entangled with long chains of the biological macromolecules when they are combined. Charged phosphate sites on the surface of bone particles, produced by washing the bone particles in basic solution, will interact with the amino groups present in many biocompatible polymers, especially those based on amino acids. The pi-orbitals on aromatic groups immobilized on a bone particle will interact with double bonds and aromatic groups of the polymer.

Additional Particulate Materials. Any type of additional components comprising inorganic materials and/or other bone substitute materials (i.e., compositions similar to natural bone such as collagen, biocompatible polymers, osteoinductive agents, other commercial bone graft products, any composite graft, etc.), may be utilized in the present invention. Inorganic materials, including but not limited to, calcium phosphate materials, and other bone substitute materials, may also be exploited for use as particulate inclusions in inventive composites. Exemplary materials utilized in accordance with the present invention include aragonite, dahlite, calcite, amorphous calcium carbonate, vaterite, weddellite, whewellite, struvite, urate, ferrihydrite, francolite, monohydrocalcite, magnetite, goethite, dentin, calcium carbonate, calcium sulfate, calcium phosphosilicate, sodium phosphate, calcium aluminate, calcium phosphate, hydroxyapatite, α-tricalcium phosphate, dicalcium phosphate, β-tricalcium phosphate, tetracalcium phosphate, amorphous calcium phosphate, octacalcium phosphate, and BIOGLASS™, a calcium phosphate silica glass available from U.S. Biomaterials Corporation. Substituted calcium phosphate phases are also contemplated for use with the invention, including but not limited to fluorapatite, chlorapatite, magnesium-substituted tricalcium phosphate, and carbonate hydroxyapatite. In certain embodiments, an inorganic material is a substituted form of hydroxyapatite. For example, hydroxyapatite may be substituted with other ions such as fluoride, chloride, magnesium, sodium, potassium, and groups such as silicates, silicon dioxides, carbonates, etc. Additional calcium phosphate phases suitable for use with the invention include those disclosed in U.S. Pat. RE 33,161 and RE 33,221 to Brown et al.; U.S. Pat. Nos. 4,880,610; 5,034,059; 5,047,031; 5,053,212; 5,129,905; 5,336,264; and 6,002,065 to Constantz et al.; U.S. Pat. Nos. 5,149,368; 5,262,166 and 5,462,722 to Liu et al.; U.S. Pat. Nos. 5,525,148 and 5,542,973 to Chow et al., U.S. Pat. Nos. 5,717,006 and 6,001,394 to Daculsi et al., U.S. Pat. No. 5,605,713 to Boltong et al., U.S. Pat. No. 5,650,176 to Lee et al., and U.S. Pat. No. 6,206,957 to Driessens et al, and biologically-derived or biomimetic materials such as those identified in Lowenstam H A, Weiner S, On Biomineralization, Oxford University Press, 1989; each of which is incorporated herein by reference.

In some embodiments, a particulate composite material may be employed to combine with inventive composites in the present invention. For example, inorganic materials such as those described above may be combined with proteins such as bovine serum albumin (BSA), collagen, or other extracellular matrix components to form a composite. In some embodiments, inorganic materials or bone-derived materials may be combined with synthetic or natural polymers to form a composite using the techniques described in our co-pending U.S. patent applications, U.S. Ser. No. 10/735,135, filed Dec. 12, 2003; U.S. Ser. No. 10/681,651, filed Oct. 8, 2003; and U.S. Ser. No. 10/639,912, filed Aug. 12, 2003, the contents of all of which are incorporated herein by reference.

As described above, particles utilized in accordance with the present invention may be optionally treated to enhance their interaction with a polymer component and/or to confer some properties to particle surface. In some embodiment, particles are modified and/or treated to react with other materials before combining with a polymer component. In some embodiments, modified particles are reacted (e.g., physically or chemically) with a polymer before combining with a polymer component (e.g., polyurethane). In some embodiments, additional particulate components comprising inorganic materials and/or other bone substitute materials are modified (e.g., surface modified).

Polymer Component

A polymer component/material used in accordance with the present invention may include a polymer, a prepolymer (e.g., an oligoment, or other macromolecules), a monomer, or any combinations thereof. In some embodiments, a polymer component/material lack one or more of materials thereof.

Polymer components useful for the preparation of provided composites include biocompatible polymer components (e.g., polymers, prepolymers, monomers, etc.), that can be of natural or synthetic origin or a combination of natural and synthetic polymers. Biodegradable polymers may be used in some embodiments. Co-polymers and/or polymer blends may also be used in some embodiments. Polymers can be designed with properties targeted for a given clinical application. According to the present invention, polyurethanes (PUR) are a useful class of biomaterials due to the fact that they can be injectable and/or moldable as a reactive liquid that subsequently cures to form a porous composite. These materials also have tunable degradation rates, which are shown to be highly dependent on the choice of polyol and isocyanate components (Hafeman et al., Pharmaceutical Research 2008; 25(10):2387-99; Storey et al., J Poly Sci Pt A: Poly Chem 1994; 32:2345-63; Skarja et al., J App Poly Sci 2000; 75:1522-34). Polyurethanes have tunable mechanical properties, which can also be enhanced with the addition of bone particles and/or other components (Adhikari et al., Biomaterials 2008; 29:3762-70; Goma et al., J Biomed Mater Res Pt A 2003; 67A(3):813-27) and exhibit elastomeric rather than brittle mechanical properties.

Polyurethanes can be made by reacting together components of a two-component composition, one of which includes a polyisocyanate (e.g., LDI, LTI) while the other includes a component having two or more hydroxyl groups (i.e., polyols) to react with the polyisocyanate. For example, U.S. Pat. No. 6,306,177, discloses a method for repairing a tissue site using polyurethanes, the content of which is incorporated by reference.

It is to be understood that by “a two-component composition” it means a composition comprising two essential types of polymer components. In some embodiments, such a composition may additionally comprise one or more other optional components.

In some embodiments, polyurethane is a polymer prepared through combination of two components (i.e., a polyisocyanate and a polyol). In some embodiments, a polyisocyanate is a molecule with two or more isocyanate moieties. In some embodiments, a polyol has at least two hydroxyl groups.

Synthesis of a polyurethane typically results from a gelling reaction between two liquid components (i.e., a polyisocyanate and a polyol), where a polyisocyanate and a polyester polyol react to form urethane bonds. In some embodiments, such reactions are illustrated below in Scheme 1. The relative rates of reactions can determine the scaffold morphology, working time, and setting time.

Exemplary gelling reactions in forming of polyurethane are shown in Scheme 1 below, where R₁, R₂ and R₃, for example, can be oligomers of caprolactone, lactide and glycolide respectively.

Gelling Reaction

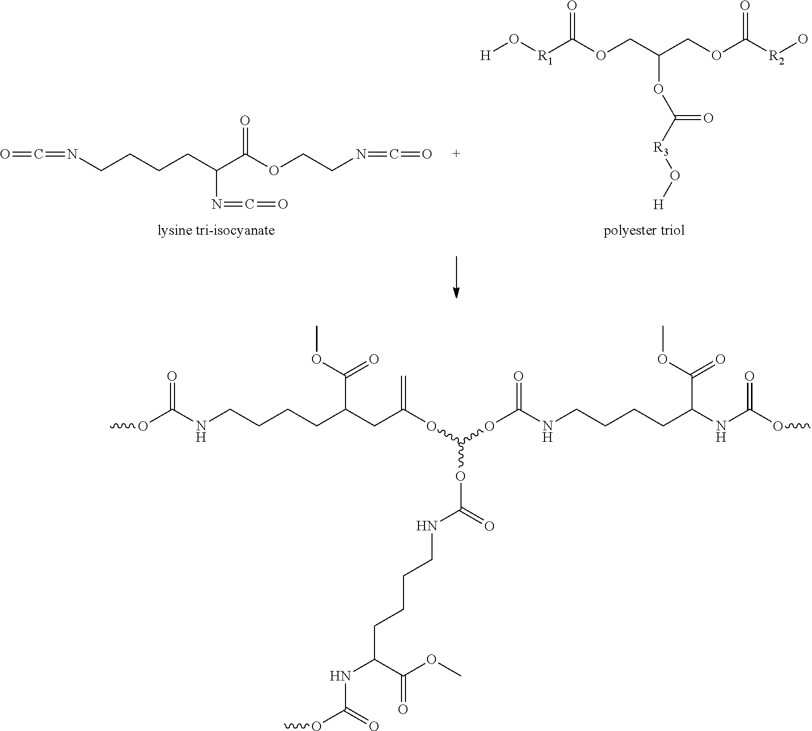

Biodegradable polyurethane scaffolds synthesized from aliphatic polyisocyanates been shown to degrade into non-toxic compounds and support cell attachment and proliferation in vitro. A variety of polyurethane polymers or components suitable for use in the present invention are known in the art, many of which are listed in commonly owned applications: U.S. Ser. No. 10/759,904 filed on Jan. 16, 2004, entitled “Biodegradable polyurethanes and use thereof” and published under No. 2005-0013793; U.S. Ser. No. 11/667,090 filed on Nov. 5, 2005, entitled “Degradable polyurethane foams” and published under No. 2007-0299151; U.S. Ser. No. 12/298,158 filed on Apr. 24, 2006, entitled “Biodegradable polyurethanes” and published under No. 2009-0221784; all of which are incorporated herein by reference. Polyurethanes described in U.S. Ser. No. 11/336,127 filed on Jan. 19, 2006 and published under No. 2006-0216323, which is entitled “Polyurethanes for Osteoimplants” and incorporated herein by reference, may be used in some embodiments of the present invention.

Polyurethanes foams may be prepared by contacting a multi-NCO-terminated component (component 1, e.g, polyisocyanate) with a hardener (component 2) that includes at least a polyol (e.g., a polyester polyol) and optionally, water, a catalyst, a stabilizer, a porogen, PEG, etc. In some embodiments, multiple polyurethanes (e.g., different structures, difference molecular weights) are used in a composite/composition of the present invention. In some embodiments, other biocompatible and/or biodegradable polymers are used with polyurethanes in accordance with the present invention. In some embodiments, biocompatible co-polymers and/or polymer blends of any combination thereof is exploited.

Polyurethanes used in accordance with the present invention can be adjusted to produce polymers having various physiochemical properties (e.g., high weight-bearing ability) and morphologies (e.g., low or non-porosity). Properties of polyurethanes are controlled by choice of the raw materials and their relative concentrations.

Polyisocyanate. A polyisocyanate component is or include a polyisocyanate and/or a polyisocyanate prepolymer. In some embodiments, polyisocyanate lacks any quasi-prepolymers.

Traditionally, polyisocyanate prepolymers (e.g., quasi-prepolymers) are prepared by contacting a polyol with an excess (typically a large excess) of a polyisocyanate. The resulting prepolymer intermediate includes an adduct of polyisocyanates and polyols solubilized in an excess of polyisocyanates. Prepolymer can, in some embodiments, be formed by using an approximately stoichiometric amount of polyisocyanates in forming a prepolymer and subsequently adding additional polyisocyanates. Prepolymers therefore exhibit both low viscosity, which facilitates processing, and improved miscibility as a result of the polyisocyanate-polyol adduct.

In the present invention, polyisocyanates or multi-isocyanate compounds are used instead of prepolymers, for example, quasi-prepolymers are disclosed in WO 2007/123536, the content of which is incorporated by reference. Polyisocanates for use in the present invention include aliphatic polyisocyanates. Exemplary aliphatic polyisocyanates include, but are not limited to, lysine diisocyanate, an alkyl ester of lysine diisocyanate (for example, the methyl ester or the ethyl ester), lysine triisocyanate, hexamethylene diisocyanate, isophorone diisocyanate (IPDI), 4,4′-dicyclohexylmethane diisocyanate (H₁₂MDI), cyclohexyl diisocyanate, 2,2,4-(2,2,4)-trimethylhexamethylene diisocyanate (TMDI), dimers prepared form aliphatic polyisocyanates, trimers prepared from aliphatic polyisocyanates and/or mixtures thereof. In some embodiments, hexamethylene diisocyanate (HDI) trimer sold as Desmodur N3300A may be a polyisocyanate utilized in the present invention.

In some embodiments, polyisocyanates used in the present invention includes approximately 10 to 55% NCO by weight (wt % NCO=100*(42/Mw)). In some embodiments, polyisocyanates include approximately 15 to 50% NCO.

In some embodiments, polyisocyantes are measured by isocyanate index, which refers to (equivalent NCO/equivalent OH)*100. In some embodiments, isocyanate index is more than 105, more than 115, or more than 125. In some embodiments, isocyanate index is 105, 115, 120, 125, 130, 140, 160, 190 or within any ranges between such index.

Polyurethane networks can, for example, then be prepared by reactive liquid molding, wherein the prepolymer is contacted with a polyester polyol to form a reactive liquid mixture (i.e., a two-component composition) which is then cast into a mold, compressed and cured. To increase reaction rates, urethane catalysts (e.g., tertiary amines) and/or elevated temperatures (60-90° C.) may be used (see, Guelcher, Tissue Engineering: Part B, 14 (1) 2008, pp 3-17). In general, the processing temperature is no greater than 60° C. In some embodiments, the processing temperature is ambient temperature (25° C.).

Polyols. Polyols utilized in accordance with the present invention can be amine- and/or hydroxyl-terminated compounds and include, but are not limited to, polyether polyols (such as polyethylene glycol (PEG or PEO), polytetramethylene etherglycol (PTMEG), polypropylene oxide glycol (PPO)); amine-terminated polyethers; polyester polyols (such as polybutylene adipate, caprolactone polyesters, castor oil); and polycarbonates (such as poly(1,6-hexanediol) carbonate). In some embodiments, polyols may be (1) molecules having multiple hydroxyl or amine functionality, such as glucose, polysaccharides, and castor oil; and (2) molecules (such as fatty acids, triglycerides, and phospholipids) that have been hydroxylated by known chemical synthesis techniques to yield polyols.

Polyols used in the present invention may be polyester polyols. In some embodiments, polyester polyols may include polyalkylene glycol esters or polyesters prepared from cyclic esters. In some embodiments, polyester polyols may include poly(ethylene adipate), poly(ethylene glutarate), poly(ethylene azelate), poly(trimethylene glutarate), poly(pentamethylene glutarate), poly(diethylene glutarate), poly(diethylene adipate), poly(triethylene adipate), poly(1,2-propylene adipate), mixtures thereof, and/or copolymers thereof. In some embodiments, polyester polyols can include, polyesters prepared from caprolactone, glycolide, D,L-lactide, mixtures thereof, and/or copolymers thereof. In some embodiments, polyester polyols can, for example, include polyesters prepared from castor-oil. When polyurethanes degrade, their degradation products can be the polyols from which they were prepared from.

In some embodiments, polyester polyols can be miscible with prepared prepolymers used in reactive liquid mixtures (i.e., two-component composition) of the present invention. In some embodiments, surfactants or other additives may be included in the reactive liquid mixtures to help homogenous mixing.

In some embodiments, particles are combined with one or more polymer components (e.g., polyisocyanate, polyols, etc.). Without being bound to any theory, it is supposed that particles (e.g., bone particles) can enhance homogenous mixing.

The glass transition temperature (Tg) of polyester polyols used in the reactive liquids to form polyurethanes can be less than 60° C., less than 37° C. (approximately human body temperature) or even less than 25° C. In addition to affecting flowability at processing conditions, Tg can also affect degradation. In general, a Tg of greater than approximately 37° C. will result in slower degradation within the body, while a Tg below approximately 37° C. will result in faster degradation.

Molecular weight of polyester polyols used in the reactive liquids to form polyurethanes can, for example, be adjusted to control the mechanical properties of polyurethanes utilized in accordance with the present invention. In that regard, using polyester polyols of higher molecular weight results in greater compliance or elasticity. In some embodiments, polyester polyols used in the reactive liquids may have a molecular weight less than approximately 1000 Da. In certain embodiments, the molecular weight may be in the range of approximately 100 to 450 Da or 50 to 1000 Da. In some embodiments, the molecular weight may be approximately in the range of approximately 450 to 1000 Da or 450 to 1200 Da. In some embodiments, molecular weight of a polyol (e.g., poly(caprolactone-co-lactide-co-glycolide)) is about 50 Da, 100 Da, 150 Da, 200 Da, 300 Da, 400 Da, 450 Da, 500 Da, 600 Da, 700 Da, 800 Da, 900 Da, 1000 Da, or within a range between any of such molecular weight.

In some embodiments, a polyester polyol comprise poly(caprolactone-co-lactide-co-glycolide), which has a molecular weight of less than about 1000 Da, less than about 600 Da, or less than 450 Da.

In some embodiments, equivalent weight of a polyol is used, which refers to molecular weight divided by functionality, and determines crosslinking efficiency. In some embodiments, triols are used in the present invention. In some embodiments, equivalent weight of polyols (e.g., triols) is less than about 333 g/eq, less than 200 g/eq, or less than 150 g/eq.

In some embodiments, polyols may include multiply types of polyols with different structures, molecular weight, properties, etc.

Additional Components. In accordance with the present invention, two-component compositions (i.e., polyisocyanates and hardener including polyols) to form composites may be used with other agents and/or catalysts.

Conventional polyurethane foams have been manufactured commercially for years. Ferrari and co-workers' in Ferrari R J, Sinner J W, Bill J C, Brucksch W F. Compounding polyurethanes: Humid aging can be controlled by choosing the right intermediate. Ind. Eng. Chem. 1958; 50(7):1041-1044, and U.S. Pat. No. 6,066,681, the disclosures of which is incorporated herein by reference, disclose methods for preparation of polyurethane foams from diisocyanates and polyester polyols. Catalysts, including organometallic compounds and tertiary amines, are added to balance the gelling (reaction of isocyanate with polyol) and blowing (reaction of isocyanate with water) reactions. Stabilizer, such as polyethersiloxanes and sulfated castor oil, are added to both emulsify the raw materials and stabilize the rising bubbles. Cell openers, such as powdered divalent salts of stearic acid, cause a local disruption of the pore structure during the foaming process, thereby yielding foams with a natural sponge structure. See Oertel G. Polyurethane Handbook. Berlin: Hanser Gardner Publications; 1994; Szycher, M, Szycher's Handbook of Polyurethanes, CRC Press, New York, N.Y., (1999), the disclosures of which are incorporated herein by reference.

Zhang et al. have found that water may be an adequate blowing agent for a lysine diisocyanate/PEG/glycerol polyurethane (see Zhang, et al., Tissue Eng. 2003 (6):1143-57) and may also be used to form porous structures in polyurethanes. Other blowing agents include dry ice or other agents that release carbon dioxide or other gases into the composite. Alternatively, or in addition, porogens (see detail discussion below) such as salts may be mixed in with reagents and then dissolved after polymerization to leave behind small voids.

Two-component compositions and/or the prepared composites used in the present invention may include one or more additional components. In some embodiments, inventive compositions and/or composites may includes, water, a catalyst (e.g., gelling catalyst, blowing catalyst, etc.), a stabilizer, a plasticizer, a porogen, a chain extender (for making of polyurethanes), a pore opener (such as calcium stearate, to control pore morphology), a wetting or lubricating agent, etc. (See, U.S. Ser. No. 10/759,904 published under No. 2005-0013793, and U.S. Ser. No. 11/625,119 published under No. 2007-0191963; both of which are incorporated herein by reference).

In some embodiments, inventive compositions and/or composites may include and/or be combined with a solid filler (e.g., carboxymethylcellulose (CMC) and hyaluronic acid (HA)). For example, when composites used in wound healing, solid fillers can help absorb excess moisture in the wounds from blood and serum and allow for proper foaming.

In certain embodiments, additional biocompatible polymers (e.g., PEG) or co-polymers can be used with compositions and composites in the present invention.

Water. Water may be a blowing agent to generate porous polyurethane-based composites. Porosity of bone/polymer composites increased with increasing water content, and biodegradation rate accelerated with decreasing polyester half-life, thereby yielding a family of materials with tunable properties that are useful in the present invention. See, Guelcher et al., Tissue Engineering, 13(9), 2007, pp 2321-2333, which is incorporated by reference.

In some embodiments, an amount of water is about 0.5, 1, 1.5, 2, 3, 4 5, 6, 7, 8, 9, parts per hundred parts (pphp) polyol. In some embodiments, water has an approximate rang of any of such amounts.

Catalyst. In some embodiments, at least one catalyst is added to form reactive liquid mixture (i.e., two-component compositions). A catalyst, for example, can be non-toxic (in a concentration that may remain in the polymer).

A catalyst can, for example, be present in two-component compositions in a concentration in the range of approximately 0.5 to 5 parts per hundred parts polyol (pphp) and, for example, in the range of approximately 0.5 to 2, or 2 to 3 pphp. A catalyst can, for example, be an amine compound. In some embodiments, catalyst may be an organometallic compound or a tertiary amine compound. In some embodiments the catalyst may be stannous octoate (an organobismuth compound), triethylene diamine, bis(dimethylaminoethyl)ether, dimethylethanolamine, dibutyltin dilaurate, and Coscat organometallic catalysts manufactured by Vertullus (a bismuth based catalyst), or any combination thereof.

Stabilizer. In some embodiments, a stabilizer is nontoxic (in a concentration remaining in the polyurethane foam) and can include a non-ionic surfactant, an anionic surfactant or combinations thereof. For example, a stabilizer can be a polyethersiloxane, a salt of a fatty sulfonic acid or a salt of a fatty acid. In certain embodiments, a stabilizer is a polyethersiloxane, and the concentration of polyethersiloxane in a reactive liquid mixture can, for example, be in the range of approximately 0.25 to 4 parts per hundred polyol. In some embodiments, polyethersiloxane stabilizer are hydrolyzable.

In some embodiments, the stabilizer can be a salt of a fatty sulfonic acid. Concentration of a salt of the fatty sulfonic acid in a reactive liquid mixture can be in the range of approximately 0.5 to 5 parts per hundred polyol. Examples of suitable stabilizers include a sulfated castor oil or sodium ricinoleicsulfonate.

Stabilizers can be added to a reactive liquid mixture of the present invention to, for example, disperse prepolymers, polyols and other additional components, stabilize the rising carbon dioxide bubbles, and/or control pore sizes of inventive composites. Although there has been a great deal of study of stabilizers, the operation of stabilizers during foaming is not completely understood. Without limitation to any mechanism of operation, it is believed that stabilizers preserve the thermodynamically unstable state of a polyurethane foam during the time of rising by surface forces until the foam is hardened. In that regard, foam stabilizers lower the surface tension of the mixture of starting materials and operate as emulsifiers for the system. Stabilizers, catalysts and other polyurethane reaction components are discussed, for example, in Oertel, Günter, ed., Polyurethane Handbook, Hanser Gardner Publications, Inc. Cincinnati, Ohio, 99-108 (1994). A specific effect of stabilizers is believed to be the formation of surfactant monolayers at the interface of higher viscosity of bulk phase, thereby increasing the elasticity of surface and stabilizing expanding foam bubbles.

Chain extender. To prepare high-molecular-weight polymers, prepolymers are chain extended by adding a short-chain (e.g., <500 g/mol) polyamine or polyol. In certain embodiments, water may act as a chain extender. In some embodiments, addition of chain extenders with a functionality of two (e.g., diols and diamines) yields linear alternating block copolymers.

Plasticizer. In some embodiments, inventive compositions and/or composites include one or more plasticizers. Plasticizers are typically compounds added to polymers or plastics to soften them or make them more pliable. According to the present invention, plasticizers soften, make workable, or otherwise improve the handling properties of polymers or composites. Plasticizers also allow inventive composites to be moldable at a lower temperature, thereby avoiding heat induced tissue necrosis during implantation. Plasticizer may evaporate or otherwise diffuse out of the composite over time, thereby allowing composites to harden or set. Without being bound to any theory, plasticizer are thought to work by embedding themselves between the chains of polymers. This forces polymer chains apart and thus lowers the glass transition temperature of polymers. In general, the more plasticizer added, the more flexible the resulting polymers or composites will be.

In some embodiments, plasticizers are based on an ester of a polycarboxylic acid with linear or branched aliphatic alcohols of moderate chain length. For example, some plasticizers are adipate-based. Examples of adipate-based plasticizers include bis(2-ethylhexyl)adipate (DOA), dimethyl adipate (DMAD), monomethyl adipate (MMAD), and dioctyl adipate (DOA). Other plasticizers are based on maleates, sebacates, or citrates such as bibutyl maleate (DBM), diisobutylmaleate (DIBM), dibutyl sebacate (DBS), triethyl citrate (TEC), acetyl triethyl citrate (ATEC), tributyl citrate (TBC), acetyl tributyl citrate (ATBC), trioctyl citrate (TOC), acetyl trioctyl citrate (ATOC), trihexyl citrate (THC), acetyl trihexyl citrate (ATHC), butyryl trihexyl citrate (BTHC), and trimethylcitrate (TMC). Other plasticizers are phthalate based. Examples of phthalate-based plasticizers are N-methyl phthalate, bis(2-ethylhexyl) phthalate (DEHP), diisononyl phthalate (DINP), bis(n-butyl)phthalate (DBP), butyl benzyl phthalate (BBzP), diisodecyl phthalate (DOP), diethyl phthalate (DEP), diisobutyl phthalate (DIBP), and di-n-hexyl phthalate. Other suitable plasticizers include liquid polyhydroxy compounds such as glycerol, polyethylene glycol (PEG), triethylene glycol, sorbitol, monacetin, diacetin, and mixtures thereof. Other plasticizers include trimellitates (e.g., trimethyl trimellitate (TMTM), tri-(2-ethylhexyl) trimellitate (TEHTM-MG), tri-(n-octyl,n-decyl) trimellitate (ATM), tri-(heptyl,nonyl) trimellitate (LTM), n-octyl trimellitate (OTM)), benzoates, epoxidized vegetable oils, sulfonamides (e.g., N-ethyl toluene sulfonamide (ETSA), N-(2-hydroxypropyl)benzene sulfonamide (HP BSA), N-(n-butyl) butyl sulfonamide (BBSA-NBBS)), organophosphates (e.g., tricresyl phosphate (TCP), tributyl phosphate (TBP)), glycols/polyethers (e.g., triethylene glycol dihexanoate, tetraethylene glycol diheptanoate), and polymeric plasticizers. Other plasticizers are described in Handbook of Plasticizers (G. Wypych, Ed., ChemTec Publishing, 2004), which is incorporated herein by reference. In certain embodiments, other polymers are added to the composite as plasticizers. In certain particular embodiments, polymers with the same chemical structure as those used in the composite are used but with lower molecular weights to soften the overall composite. In other embodiments, different polymers with lower melting points and/or lower viscosities than those of the polymer component of the composite are used.

In some embodiments, polymers used as plasticizer are poly(ethylene glycol) (PEG). PEG used as a plasticizer is typically a low molecular weight PEG such as those having an average molecular weight of 1000 to 10000 g/mol, for example, from 4000 to 8000 g/mol. In certain embodiments, PEG 4000, PEG 5000, PEG 6000, PEG 7000, PEG 8000 or combinations thereof are used in inventive composites. For example, plasticizer (PEG) is useful in making more moldable composites that include poly(lactide), poly(D,L-lactide), poly(lactide-co-glycolide), poly(D,L-lactide-co-glycolide), or poly(caprolactone). Plasticizer may comprise 1-40% of inventive composites by weight. In some embodiments, the plasticizer is 10-30% by weight. In some embodiments, the plasticizer is approximately 10%, 15%, 20%, 25%, 30% or 40% by weight. In other embodiments, a plasticizer is not used in the composite. For example, in some polycaprolactone-containing composites, a plasticizer is not used.

In some embodiments, inert plasticizers may be used. In some embodiments, a plasticizer may not be used in the present invention.

Porogen. Porosity of inventive composites may be accomplished using any means known in the art. Exemplary methods of creating porosity in a composite include, but are not limited to, particular leaching processes, gas foaming processing, supercritical carbon dioxide processing, sintering, phase transformation, freeze-drying, cross-linking, molding, porogen melting, polymerization, melt-blowing, and salt fusion (Murphy et al., Tissue Engineering 8(1):43-52, 2002; incorporated herein by reference). For a review, see Karageorgiou et al., Biomaterials 26:5474-5491, 2005; incorporated herein by reference. Porosity may be a feature of inventive composites during manufacture or before implantation, or porosity may only be available after implantation. For example, a implanted composite may include latent pores. These latent pores may arise from including porogens in the composite.

Porogens may be any chemical compound that will reserve a space within the composite while the composite is being molded and will diffuse, dissolve, and/or degrade prior to or after implantation or injection leaving a pore in the composite. Porogens may have the property of not being appreciably changed in shape and/or size during the procedure to make the composite moldable. For example, a porogen should retain its shape during the heating of the composite to make it moldable. Therefore, a porogen does not melt upon heating of the composite to make it moldable. In certain embodiments, a porogen has a melting point greater than about 60° C., greater than about 70° C., greater than about 80° C., greater than about 85° C., or greater than about 90° C.

Porogens may be of any shape or size. A porogen may be spheroidal, cuboidal, rectangular, elonganted, tubular, fibrous, disc-shaped, platelet-shaped, polygonal, etc. In certain embodiments, the porogen is granular with a diameter ranging from approximately 100 microns to approximately 800 microns. In certain embodiments, a porogen is elongated, tubular, or fibrous. Such porogens provide increased connectivity of pores of inventive composite and/or also allow for a lesser percentage of the porogen in the composite.

Amount of porogens may vary in inventive composite from 1% to 80% by weight. In certain embodiments, the plasticizer makes up from about 5% to about 80% by weight of the composite. In certain embodiments, a plasticizer makes up from about 10% to about 50% by weight of the composite. Pores in inventive composites are thought to improve the osteoinductivity or osteoconductivity of the composite by providing holes for cells such as osteoblasts, osteoclasts, fibroblasts, cells of the osteoblast lineage, stem cells, etc. Pores provide inventive composites with biological in growth capacity. Pores may also provide for easier degradation of inventive composites as bone is formed and/or remodeled. In some embodiments, a porogen is biocompatible.

A porogen may be a gas, liquid, or solid. Exemplary gases that may act as porogens include carbon dioxide, nitrogen, argon, or air. Exemplary liquids include water, organic solvents, or biological fluids (e.g., blood, lymph, plasma). Gaseous or liquid porogen may diffuse out of the osteoimplant before or after implantation thereby providing pores for biological in-growth. Solid porogens may be crystalline or amorphous. Examples of possible solid porogens include water soluble compounds. Exemplary porogens include carbohydrates (e.g., sorbitol, dextran (poly(dextrose)), starch), salts, sugar alcohols, natural polymers, synthetic polymers, and small molecules.

In some embodiments, carbohydrates are used as porogens in inventive composites. A carbohydrate may be a monosaccharide, disaccharide, or polysaccharide. The carbohydrate may be a natural or synthetic carbohydrate. In some embodiments, the carbohydrate is a biocompatible, biodegradable carbohydrate. In certain embodiments, the carbohydrate is a polysaccharide. Exemplary polysaccharides include cellulose, starch, amylose, dextran, poly(dextrose), glycogen, etc.

In certain embodiments, a polysaccharide is dextran. Very high molecular weight dextran has been found particularly useful as a porogen. For example, the molecular weight of the dextran may range from about 500,000 g/mol to about 10,000,000 g/mol, preferably from about 1,000,000 g/mol to about 3,000,000 g/mol. In certain embodiments, the dextran has a molecular weight of approximately 2,000,000 g/mol. Dextrans with a molecular weight higher than 10,000,000 g/mol may also be used as porogens. Dextran may be used in any form (e.g., particles, granules, fibers, elongated fibers) as a porogen. In certain embodiments, fibers or elongated fibers of dextran are used as a porogen in inventive composites. Fibers of dextran may be formed using any known method including extrusion and precipitation. Fibers may be prepared by precipitation by adding an aqueous solution of dextran (e.g., 5-25% dextran) to a less polar solvent such as a 90-100% alcohol (e.g., ethanol) solution. The dextran precipitates out in fibers that are particularly useful as porogens in the inventive composite. Once the composite with dextran as a porogen is implanted into a subject, the dextran dissolves away very quickly. Within approximately 24 hours, substantially all of dextran is out of composites leaving behind pores in the osteoimplant composite. An advantage of using dextran in a composite is that dextran exhibits a hemostatic property in extravascular space. Therefore, dextran in a composite can decrease bleeding at or near the site of implantation.

Small molecules including pharmaceutical agents may also be used as porogens in the inventive composites. Examples of polymers that may be used as plasticizers include poly(vinyl pyrollidone), pullulan, poly(glycolide), poly(lactide), and poly(lactide-co-glycolide). Typically low molecular weight polymers are used as porogens. In certain embodiments, a porogen is poly(vinyl pyrrolidone) or a derivative thereof. Plasticizers that are removed faster than the surrounding composite can also be considered porogens.

Components to be Delivered

Composites of the present invention may include one or more components to be delivered when implanted. Representative such composites may include biomolecules, small molecules, bioactive agents, etc. In some embodiments, such materials promote bone growth and connective tissue regeneration, and/or to accelerate healing. Particular examples of materials that can be incorporated include chemotactic factors, angiogenic factors, bone cell inducers and stimulators, including the general class of cytokines such as the TGF-β superfamily of bone growth factors, the family of bone morphogenic proteins, osteoinductors, and/or bone marrow or bone forming precursor cells, isolated using standard techniques. Sources and amounts of such materials that can be included are known to those skilled in the art.

Biologically active materials, comprising biomolecules, small molecules, and/or bioactive agents may also be included in inventive composites to, for example, stimulate particular metabolic functions, recruit cells, or reduce inflammation. For example, nucleic acid vectors, including plasmids and viral vectors, that will be introduced into the patient's cells and cause the production of growth factors such as bone morphogenetic proteins may be included in a composite. Biologically active agents include, but are not limited to, antiviral agent, antimicrobial agent, antibiotic agent, amino acid, peptide, protein, glycoprotein, lipoprotein, antibody, steroidal compound, antibiotic, antimycotic, cytokine, vitamin, carbohydrate, lipid, extracellular matrix, extracellular matrix component, chemotherapeutic agent, cytotoxic agent, growth factor, anti-rejection agent, analgesic, anti-inflammatory agent, viral vector, protein synthesis co-factor, hormone, endocrine tissue, synthesizer, enzyme, polymer-cell scaffolding agent with parenchymal cells, angiogenic drug, collagen lattice, antigenic agent, cytoskeletal agent, mesenchymal stem cells, bone digester, antitumor agent, cellular attractant, fibronectin, growth hormone cellular attachment agent, immunosuppressant, nucleic acid, surface active agent, hydroxyapatite, and penetraction enhancer. Additional exemplary substances include chemotactic factors, angiogenic factors, analgesics, antibiotics, anti-inflammatory agents, bone morphogenic proteins, and other growth factors that promote cell-directed degradation or remodeling of the polymer phase of the composite and/or development of new tissue (e.g., bone). RNAi or other technologies may also be used to reduce the production of various factors.

In some embodiments, inventive composites include antibiotics. Antibiotics may be bacteriocidial or bacteriostatic. An anti-microbial agent may be included in composites. For example, anti-viral agents, anti-protazoal agents, anti-parasitic agents, etc. may be include in composites. Other suitable biostatic/biocidal agents include antibiotics, povidone, sugars, and mixtures thereof. Exemplary antibiotics include, but not limit to, Amikacin, Gentamicin, Kanamycin, Neomycin, Netilmicin, Streptomycin, Tobramycin, Paromomycin, Geldanamycin, Herbimycin, Loravabef, etc. (See, The Merck Manual of Medical Information—Home Edition, 1999).

Inventive composites may be seeded with cells. In some embodiments, a patient's own cells are obtained and used in inventive composites. Certain types of cells (e.g., osteoblasts, fibroblasts, stem cells, cells of the osteoblast lineage, etc.) may be selected for use in the composite. Cells may be harvested from marrow, blood, fat, bone, muscle, connective tissue, skin, or other tissues or organs. In some embodiments, a patient's own cells may be harvested, optionally selected, expanded, and used in the inventive composite. In other embodiments, a patient's cells may be harvested, selected without expansion, and used in the inventive composite. Alternatively, exogenous cells may be employed. Exemplary cells for use with the invention include mesenchymal stem cells and connective tissue cells, including osteoblasts, osteoclasts, fibroblasts, preosteoblasts, and partially differentiated cells of the osteoblast lineage. Cells may be genetically engineered. For example, cells may be engineered to produce a bone morphogenic protein.

In some embodiments, a component to be delivered by an inventive composite may be or include a complex material. For example, a component to be delivered may be or comprise a biomolecule (e.g., a protein) encapsulated in a polymeric microsphere or nanoparticles. In certain embodiments, BMP-2 encapsulated in PLGA microspheres may be embedded in a bone/polyurethane composite used in accordance with the present invention. Sustained release of BMP-2 can be achieved due to the diffusional barriers presented by both the PLGA and Polyurethane of the inventive composite. Thus, release kinetics of growth factors (e.g., BMP-2) can be tuned by varying size of PLGA microspheres and porosity of polyurethane composite.

In some embodiments, composites of the present invention can include different enzymes, for example to enhance biodegradation in vivo. Examples of suitable enzymes or similar reagents are proteases or hydrolases with ester-hydrolyzing capabilities. Such enzymes include, but are not limited to, proteinase K, bromelaine, pronase E, cellulase, dextranase, elastase, plasmin streptokinase, trypsin, chymotrypsin, papain, chymopapain, collagenase, subtilisin, chlostridopeptidase A, ficin, carboxypeptidase A, pectinase, pectinesterase, an oxireductase, an oxidase, or the like. The inclusion of an appropriate amount of such a degradation enhancing agent can be used to regulate implant duration.

In some embodiments, components to be delivered may be covalently bonded to a component of composites; in some embodiments, a component to be delivered may be not covalently bonded to composites. In some embodiments, covalently linked embodiments, a components be to delivered may be covalently linked to a particular component and/or to a polymer component to further being combined to generate a composite. In some embodiments, covalently linked embodiments, a component to be delivered is covalently linked to a composite after combination of polymer and particle components. In some embodiments, components to be delivered are covalently linked to composites by way of a silane linker. As discussed above, for example, silane coupling agents having amine, carboxyl, hydroxyl, or mercapto groups may be attached to the bone particles through the silane and then to reactive groups on a biomolecule, small molecule, or bioactive agent.

Preparation of Composites

In general, inventive composites are prepared by combining particle components, polymer components, and optionally any additional components. To form inventive composites, particles as discussed herein may be combined with a reactive liquid (i.e., a two-component composition) thereby forming a naturally flowable or moldable composite or a composite that can be made injectable or moldable. Alternatively, particles may be combined with polyisocyanate or polyols first and then combined with other components.

In some embodiments, particles may be combined first with a hardener that includes polyols, water, catalysts and optionally a solvent, a diluent, a stabilizer, a porogen, a plasticizer, etc., and then combined with a polyisocyanate and/or a prepolymer. In some embodiments, a hardener (e.g., a polyol, and optionally a catalyst) may be mixed with a polyisocyanates, followed by addition of particles. In some embodiments, in order to enhance storage stability of two-component compositions, the two (liquid) component process is modified to an alternative three (liquid)-component process wherein a catalyst is dissolved in a solution separating from reactive polyols. For example, polyester polyols may be first mixed with a solution of a catalyst, followed by addition of bone particles, and finally addition of a NCO-terminated component.

In some embodiments, additional components or components to be delivered are combined with a reactive liquid prior to injection. In some embodiments, they are combined with one of polymer precursors (i.e., polyisocyanates and polyols) prior to mixing the precursors in forming of a reactive liquid/paste.

In some embodiments, catalysts can be used to assist forming composites with porosity in a wide range. In general, the more blowing catalyst used, the high porosity of inventive composites may be achieved. In certain embodiments, surprisingly, surface demineralized bone particles may have a dramatic effect on the porosity. Without being bound to any theory, it is believed that the lower porosities achieved with surface demineralized bone particles result from adsorption of water to the hygroscopic demineralized layer on the surface of bones.

Polymers and particles may be combined in a one-shot process. For example, a homogenous mixture of polymers and/or polymer precursors (e.g., polyisocyanates, polyols, etc.) may be mixed together. In some embodiments, particles are combined with polymer components in a one-shot process. In some embodiments, mixing is done at ambient or elevated temperatures.

Alternatively or additionally, mixing of polymers with particles is done under pressure. In some embodiments, compression pressure utilized in accordance with the present application is more than 5,000 psi, more than 30,000 psi, or more than 50,000 psi.

At elevated temperatures, a process may also be accomplished without pressure. In some embodiments, polymers or precursors are not held at a temperature of greater than approximately 60° C. for a significant time during mixing to prevent thermal damage to any biological component (e.g., growth factors or cells) of a composite. In some embodiments, temperature is not a concern because particles and polymer precursors (e.g, polyurethane precursors) used in the present invention have a low reaction exotherm.

Alternatively or additionally, particles may be mixed or folded into a polymer softened by heat or a solvent. Alternatively, a moldable polymer may be formed into a sheet that is then covered with a layer of particles. Particles may then be forced into the polymer sheet using pressure. In another embodiment, particles are individually coated with polymers or polymer precursors, for example, using a tumbler, spray coater, or a fluidized bed, before being mixed with a larger quantity of polymer. This facilitates even coating of the particles and improves integration of the particles and polymer component of the composite.

After combination with particles, polymers or polymer components may be further modified by further cross-linking or polymerization to form a composite in which the polymer is covalently linked to the particles. In some embodiments, composition hardens in a solvent-free condition. In some embodiments, compositions are a polymer/solvent mixture that hardens when a solvent is removed (e.g., when a solvent is allowed to evaporate or diffuse away). Exemplary solvents include but are not limited to alcohols (e.g., methanol, ethanol, propanol, butanol, hexanol, etc.), water, saline, DMF, DMSO, glycerol, and PEG. In certain embodiments, a solvent is a biological fluid such as blood, plasma, serum, marrow, etc. In certain embodiments, an inventive composite is heated above the melting or glass transition temperature of one or more of its components and becomes set after implantation as it cools. In certain embodiments, an inventive composite is set by exposing a composite to a heat source, or irradiating it with microwaves, IR rays, or UV light. Particles may also be mixed with a polymer that is sufficiently pliable to combine with the particles but that may require further treatment, for example, combination with a solvent or heating, to become a flowable or moldable composite/composition. For example, a composition may be combined and injection molded, injected, extruded, laminated, sheet formed, foamed, or processed using other techniques known to those skilled in the art. In some embodiments, compression molding methods, in which polymer precursors (e.g., polyisocyanate, a polyol) are separately charged into a mold under precisely defined conditions, may be employed. For example, bone particles may be added to a precursor, or it may be separately charged into a mold and precursor materials added afterwards. Careful control of relative amounts of various components and reaction conditions may be desired to limit the amount of unreacted material in a composite. Post-cure processes known to those skilled in the art may also be employed. A partially polymerized polyurethane precursor may be more completely polymerized or cross-linked after combination with hydroxylated or aminated materials or included materials (e.g., a particulate, any components to deliver, etc.).

In some embodiments, particles may be mixed with a polymer precursor according to standard composite processing techniques. For example, regularly shaped particles may simply be suspended in a precursor. A polymer precursor may be mechanically stirred to distribute the particles or bubbled with a gas, preferably one that is oxygen- and moisture-free. Once components of a composition are mixed, it may be desirable to store it in a container that imparts a static pressure to prevent separation of the particles and the polymer precursor, which may have different densities. In some embodiments, distribution and particle/polymer ratio may be optimized to produce at least one continuous path through a composite along particles.

In some embodiments, it may be desirable to infiltrate a polymer or polymer precursor into vascular and/or interstitial structure of bone particles or into bone-derived tissues. Vascular structure of bone includes such structures such as osteocyte lacunae, Haversian canals, Volksmann's canals, canaliculi and similar structures. Interstitial structure of bone particles includes spaces between trabeculae and similar features. Many of monomers and precursors (e.g., polyisocyanate, polyols) suggested for use with the invention are sufficiently flowable to penetrate through the channels and pores of trabecular bone. Some may even penetrate into trabeculae or into mineralized fibrils of cortical bone. Thus, it may be necessary to incubate bone particles in polyurethane precursors for a period of time to accomplish infiltration. In certain embodiments, polyurethane itself is sufficiently flowable that it can penetrate channels and pores of bone. In certain embodiments, polyurethane may also be heated or combined with a solvent to make it more flowable for this purpose. Other ceramic materials and/or other bone-substitute materials employed as a particulate phase may also include porosity that can be infiltrated as described herein.

Inventive composites utilized in the present invention may include practically any ratio of polymer components (e.g., polyurethane) and particulate components (e.g., bone particles), for example, between about 5 wt % and about 95 wt % bone particles. In some embodiments, composites may include about 40 wt % to about 45 wt % particles, about 45 wt % to about 50 wt % particles or about 50 wt % to about 55 wt % particles. In some embodiments, composites may include about 55 wt % to about 70 wt % particles. In some embodiments, composites may include about 70 wt % to about 90 wt % particles. In some embodiments, composites may include at least approximately 40 wt %, 45 wt %, 50 wt %, or 55 wt % of particles. In certain embodiments, such weight percentages refer to weight of bone particles and other particulates such as calcium phosphate.

In some embodiments, composites may include at least approximately 40 vol %, 45 vol %, 50 vol %, or 57 vol % of particles. In some embodiments, a volume percentage of particles in composite in accordance with the present invention may be about 40 vol %, 45 vol %, 50 vol %, 57 vol %, 60 vol %, 65 vol %, 70 vol % or between any volume percentages of above. In some embodiments, flowable or moldable composites in accordance with the present invention may have a volume percentage (fraction) of at least approximately 57 vol % of bone particles and/or other particulate materials (e.g., calcium phosphate). In some embodiments, volume percentages (fractions) of bone particles and/or other particulate materials in porous composites in the present invention are less than 64 vol %. In certain embodiments, for a certain volume percentage, corresponding weight percentage of bone particles and/or other particulate materials varies depending on density of particulate components.

Desired proportions may depend on factors such as shape and size of particles, how evenly polymer is distributed among particles, desired flowability of composites, desired handling of composites, desired moldability of composites, and mechanical and degradation properties of composites. Proportions of polymers and particles can influence various characteristics of the composite, for example, its mechanical properties, including fatigue strength, the degradation rate, and the rate of biological incorporation. In addition, the cellular response to the composite will vary with the proportion of polymer and particles. In some embodiments, desired proportion of particles may be determined not only by the desired biological properties of a flowable or moldable composite but by the desired mechanical properties of such composites. That is, an increased proportion of particles will increase the viscosity of the composite, making it more difficult to inject or mold. A larger proportion of particles having a wide size distribution may give similar properties to a mixture having a smaller proportion of more evenly sized particles.

Surface modification of components (e.g., particular components) can be used to adjust properties of provided composites. In some embodiments, defatted, demineralized particles, and/or surface demineralized particles are used to combined with polymer components. In some embodiments, defatted bone particles are used in the present invention and bonds well with polymer components, thereby improving the mechanical properties of provided composites. Without being bound to any theory, removing fat covered on bone particles enhance interfacial binding between bone and polymer (e.g, isocyanante). In some embodiments, surface demineralized bone particles are used in the present invention and bonds well with polymer components. Without being bound to any theory, it is believed that surface modification of particles increases the reactivity toward isocyanates, but in some embodiments, such enhanced properties does not translate to improved mechanical properties of provided composites.

Inventive composites of the present invention can exhibit high degrees of weight-bearing abilities over a wide range. In some embodiments, provided composites have wet compressive strength of more than 50 MPa, more than 100 MPa, or more than 150 MPa. In some embodiments, provided composites have wet compressive strength of 107-172 MPa. In some embodiments, provided composites have wet compressive modulus of more than 1 GPa, more than 3 GPa, or more than 5 GPa. In some embodiments, composites has wet compressive modulus of 3-6 GPa.

After implantation, inventive composites are allowed to remain at the site providing the strength desired while at the same time promoting healing of the bone and/or bone growth. Polyurethane of composites may be degraded or be resorbed as new bone is formed at the implantation site. Polymer may be resorbed over approximately 1 month to approximately 1 years. Composites may start to be remodeled in as little as a week as the composite is infiltrated with cells or new bone in-growth. A remodeling process may continue for weeks, months, or years. For example, polyurethanes used in accordance with the present invention may be resorbed within about 4-8 weeks, 2-6 months, or 6-12 months. A degradation rate is defined as the mass loss as a function of time, and it can be measured by immersing the sample in phosphate buffered saline or medium and measuring the sample mass as a function of time.

One skilled in the art will recognize that standard experimental techniques may be used to test these properties for a range of compositions to optimize a composite for a desired application. For example, standard mechanical testing instruments may be used to test the compressive strength and stiffness of composites. Cells may be cultured on composites for an appropriate period of time, and metabolic products and amount of proliferation (e.g., the number of cells in comparison to the number of cells seeded) may be analyzed. Weight change of composites may be measured after incubation in saline or other fluids. Repeated analysis will demonstrate whether degradation of a composite is linear or not, and mechanical testing of incubated materials will show changes in mechanical properties as a composite degrades. Such testing may also be used to compare enzymatic and non-enzymatic degradation of a composite and to determine levels of enzymatic degradation. A composite that is degraded is transformed into living bone upon implantation.

Use and Application of Composites

As discussed above, polymers or polymer precursors, and particles may be supplied separately, e.g., in a kit, and mixed immediately prior to implantation, injection or molding. A kit may contain a preset supply of bone particles having, e.g., certain sizes, shapes, and levels of demineralization. Surface of bone particles may have been optionally modified using one or more of techniques described herein. Alternatively, a kit may provide several different types of particles of varying sizes, shapes, and levels of demineralization and that may have been chemically modified in different ways. A surgeon or other health care professional may also combine components in a kit with autologous tissue derived during surgery or biopsy. For example, a surgeon may want to include autogenous tissue or cells, e.g., bone marrow or bone shavings generated while preparing a implant site, into a composite (see more details in co-owned U.S. Pat. No. 7,291,345 and U.S. Ser. No. 11/625,119 published under No. 2007-0191963; both of which are incorporated herein by reference).

Composites of the present invention may be used in any of a wide variety of clinical applications. A method of preparing and using polyurethanes for orthopedic applications utilized in the present invention may include the steps of providing a curable bone/polyurethane composition, mixing parts of a composition, and curing a composition in a tissue site wherein a composition is sufficiently flowable to permit injection by minimally invasive techniques. In some embodiments, a flowable composition to inject may be pressed by hand or machine. In some embodiments, a moldable composition may be pre-molded and implanted into a target site. Flowable or moldable composites/compositions utilized in the present invention may be processed (e.g., mixed, pressed, molded, etc.) by hand or machine. In some embodiments, composites are compression molded under a high pressure.

Inventive composites and/or compositions may be used as moldable materials with exhibiting high mechanical strength (i.e., weight-bearing). In some embodiments, inventive composites and/or compositions may be used as moldable materials. For example, compositions (e.g., prepolymers, precursors, monomers, reactive liquids/pastes, polymers, particles, additional components, etc.) in the present invention can be pre-molded into pre-determined shapes. Upon implantation, the pre-molded composite may further cure in situ and provide mechanical strength (i.e., load-bearing). A few examples of potential applications are discussed in more detail below.

In some embodiments, compositions and/or composites of the present invention may be used as a bone void filler. Bone fractures and defects, which result from trauma, injury, infection, malignancy or developmental malformation can be difficult to heal in certain circumstances. If a defect or gap is larger than a certain critical size, natural bone is unable to bridge or fill the defect or gap. These are several deficiencies that may be associated with the presence of a void in a bone. Bone void may compromise mechanical integrity of bone, making bone potentially susceptible to fracture until void becomes ingrown with native bone. Accordingly, it is of interest to fill such voids with a substance which helps voids to eventually fill with naturally grown bone. Open fractures and defects in practically any bone may be filled with composites according to various embodiments without the need for periosteal flap or other material for retaining a composite in fracture or defect. Even where a composite is not required to bear weight, physiological forces will tend to encourage remodeling of a composite to a shape reminiscent of original tissues.

Many orthopedic, periodontal, neurosurgical, oral and maxillofacial surgical procedures require drilling or cutting into bone in order to harvest autologous implants used in procedures or to create openings for the insertion of implants. In either case voids are created in bones. In addition to all the deficiencies associated with bone void mentioned above, surgically created bone voids may provide an opportunity for incubation and proliferation of any infective agents that are introduced during a surgical procedure. Another common side effect of any surgery is ecchymosis in surrounding tissues which results from bleeding of the traumatized tissues. Finally, surgical trauma to bone and surrounding tissues is known to be a significant source of post-operative pain and inflammation. Surgical bone voids are sometimes filled by the surgeon with autologous bone chips that are generated during trimming of bony ends of a graft to accommodate graft placement, thus accelerating healing. However, the volume of these chips is typically not sufficient to completely fill the void. Composites and/or compositions of the present invention, for example composites comprising anti-infective and/or anti-inflammatory agents, may be used to fill surgically created bone voids.

Inventive composites and/or compositions may be administered to a subject in need thereof using any technique known in the art. A subject is typically a patient with a disorder or disease related to bone. In certain embodiments, a subject has a bony defect such as a fracture. In some embodiment, a subject is typically a mammal although any animal with bones may benefit from treatment with the inventive composite. In certain embodiments, a subject is a vertebrate (e.g., mammals, reptiles, fish, birds, etc.). In certain embodiments, a subject is a human. In other embodiments, the subject is a domesticated animal such as a dog, cat, horse, etc. Any bone disease or disorder may be treated using inventive composites/compositions including genetic diseases, congenital abnormalities, fractures, iatrogenic defects, bone cancer, bone metastases, inflammatory diseases (e.g., rheumatoid arthritis), autoimmune diseases, metabolic diseases, and degenerative bone disease (e.g., osteoarthritis). In certain embodiments, inventive osteoimplant composites are formulated for repair of a simple fracture, compound fracture, or non-union; as an external fixation device or internal fixation device; for joint reconstruction, arthrodesis, arthroplasty, or cup arthroplasty of hips; for femoral or humeral head replacement; for femoral head surface replacement or total joint replacement; for repair of vertebral column, spinal fusion or internal vertebral fixation; for tumor surgery; for deficit filling; for discectomy; for laminectomy; for excision of spinal tumors; for an anterior cervical or thoracic operation; for the repairs of a spinal injury; for scoliosis, for lordosis or kyphosis treatment; for intermaxillary fixation of a fracture; for mentoplasty; for temporomandibular joint replacement; for alveolar ridge augmentation and reconstruction; as an inlay osteoimplant; for implant placement and revision; for sinus lift; for a cosmetic procedure; and, for the repair or replacement of the ethmoid, frontal, nasal, occipital, parietal, temporal, mandible, maxilla, zygomatic, cervical vertebra, thoracic vertebra, lumbar vertebra, sacrum, rib, sternum, clavicle, scapula, humerus, radius, ulna, carpal bones, metacarpal bones, phalanges, ilium, ischium, pubis, femur, tibia, fibula, patella, calcaneus, tarsal bones, or metatarsal bones, and for repair of bone surrounding cysts and tumors.

Composites and/or compositions of the present invention can be used as bone void fillers either alone or in combination with one or more other conventional devices, for example, to fill the space between a device and bone. Examples of such devices include, but are not limited to, bone fixation plates (e.g., cranofacial, maxillofacial, orthopedic, skeletal, and the like); screws, tacks, clips, staples, nails, pins or rods, anchors (e.g., for suture, bone, and the like), scaffolds, scents, meshes (e.g., rigid, expandable, woven, knitted, weaved, etc), sponges, implants for cell encapsulation or tissue engineering, drug delivery (e.g., carriers, bone ingrowth induction catalysts such as bone morphogenic proteins, growth factors (e.g., PDGF, VEGF and BMP-2), peptides, antivirals, antibiotics, etc), monofilament or multifilament structures, sheets, coatings, membranes (e.g., porous, microporous, resorbable, etc), foams (e.g., open cell or close cell), screw augmentation, cranial, reconstruction, and/or combinations thereof.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Materials

Lysine triisocyanate (LTI) was purchased from Kyowa Hakko (New York, N.Y.). Tegoamin 33, a tertiary amine catalyst, was received from Goldschmidt (Hopewell, Va.). Glycerol, stannous octoate, and ε-caprolactone were purchased from Sigma-Aldrich (St Louis, Mo.), and glycolide and DL-lactide were supplied by Polysciences (Warrington, Pa.). Rabbit allograft mineralized bone particles (MBP, 481 μm mean particle size) were received as a gift from Osteotech, Inc. (Eatontown, N.J.).

Synthesis of Polyester Triols

Polyester triols were synthesized using published techniques.[46, 58] Briefly, the appropriate amounts of glycerol starter and ε-caprolactone, glycolide, and DL-lactide monomers were mixed under argon at 140° C. for 30 h. When the reaction was complete, the polyester triol was cooled, washed with hexane, and dried at 80° C. under vacuum. The backbone of the polyester triols comprised 60% ε-caprolactone, 30% glycolide, and 10% DL-lactide (6C3G1L). Molecular weights of 300 g/mol (6C3G1L300) and 600 g/mol (6C3G1L600) were synthesized for this study.

Preparation of Surface-Demineralized Bone Particles (SDBP).

Surface demineralized bone particles (SDBP) were prepared using published methods.[59] MBP was sonicated in 0.1 M hydrochloric acid for 2.5 minutes followed by saturation in 2.5% trypsin at 37° C. overnight. Sonication in hydrochloric acid was repeated for the same time period followed by 48 hours of saturation in 2.5% trypsin. The resulting SDBP was rinsed thoroughly with DI water and lyophilized for 48 hours.

Characterization of Reactivity of Allograft Bone Particles by FITC Labeling.

Approximately 10 mg of rabbit MBP or SDBP was added to 2 mL centrifuge tubes along with 1 mL of borate buffer. A solution of FITC, in borate buffer, was prepared to yield a concentration of 7 mg/mL, and 0.1 mL of the resulting solution was added to each tube. As a control, only borate buffer was added to three of the MBP samples. The tubes were placed on a hematology mixer for 1 hour. The tubes were centrifuged at 2500 rpm for 3 minutes to remove excess FITC from each tube, and the MBP was washed thrice with borate buffer solution. The MBP was transferred to a 96 well plate by suspending it in a solution of 0.1 mL of borate buffer. The fluorescence of each well was read using a FL600 microplate reader at an excitation of 495 nm and an emission at 525 nm. The fluorescence was read at a sensitivity of 75.

Scanning Electron Microscopy.

Rabbit MBP (approximately 5 mg) was mounted on a SEM pin stub mount and sputter-coated for 60 seconds using a Cressington Q108 sputter coater, which deposited gold at a 30 mA current. A Hitachi S-4200 scanning electron microscope was used to acquire images at a voltage of 1 kV.

Fabrication of MBP/PUR Composites.

The components of the composite were mixed using a one-shot method, wherein the appropriate amounts of Tegoamin 33, polyester triol, MBP, and LTI were added to a 10 mL cup and mixed using a Hauschild SpeedMixer (FlackTek, Inc., Landrum, S.C.). The mixture speed was gradually ramped to 3300 rpm for one minute and mixing continued at 3300 rpm for 30 s. All composites incorporated 79.0 wt % (66.2 vol %) allograft bone. The reactive paste was transferred to a cylindrical mold, compressed to approximately 63,000 lbf for 50 minutes, de-molded to yield a green cylinder (6.1 mm diameter), and cured at 37° C. for twelve hours in a vacuum oven. The four formulations listed in Table 1 were designed to investigate the effects of surface demineralization and polyester triol molecular weight on mechanical properties and remodeling in a rabbit distal femoral plug model.

TABLE 1
Bone/polymer composite formulations. MBP denotes mineralized bone
particles, SDBP denotes surface-demineralized bone particles, and
300 and 600 denote the molecular weight (g/mol) of the polyester
triols used to prepare the composites. Bovine and rabbit allograft
bone particles were supplied by Osteotech, Inc.

| Composite | MBP300 | SDBP300 | MBP600 | SDBP600 |
|---|---|---|---|---|
| Polyol | 6C3G1L300 | 6C3G1L300 | 6C3G1L600 | 6C3G1L600 |
| Filler | MBP | SDBP | MBP | SDBP |

Infrared Spectroscopy.

Potassium bromide pellets of both composites and MBP were produced using a pellet die assembly. A thin disc from the composite rods was cut using a Buehler diamond embedded circular saw, and approximately 8 mg of the composite and MBP were ground using mortar and pestle followed by the addition of 200 mg of potassium bromide. The resulting mixture was then pressed into a pellet. A Bruker Tensor 27 FTIR was used to scan each sample.

Mechanical and Swelling Properties.

Cylindrical PUR/MBP rods, approximately 6.3×12.6 mm (n=3), were fabricated by compression molding. The rods were hydrated in PBS 24 hours prior to testing. The cylinders were placed between two fixed compression platens of an MTS 898 equipped with a 13 kN load cell, pre-loaded to approximately 12 N, and subsequently loaded at 24 mm/min until failure. Swelling data were calculated from the dry and wet mass of the composites after 24 h incubation time in PBS (a time-course study showed that the composites attained equilibrium by 24 h swelling time). One-way ANOVA with bonferroni correction (p<0.05) was used for evaluation of statistical significance for both swelling and mechanical properties data.

Animal Study

Six New Zealand White (NZW) rabbits weighing between 3.8 and 4.1 kg were used in this study. All surgical and care procedures were carried out under aseptic conditions per the approved IACUC protocol. The MBP/PUR composite plugs were irradiated using a dose of approximately 25 kGY. Glycopyrrolate was administered at 0.01 mg/kg IM followed by ketamine at 40 mg/kg IM. Bilateral defects of approximately 6.1 mm diameter by 11 mm in depth were drilled in the metaphysis of the distal femurs of each rabbit. MBP/PUR plugs from each treatment group (n=3) were subsequently inserted into each defect. Treatment groups for each composite were dispersed randomly among the rabbits. The rabbits were euthanized after six weeks using Fatal-plus (2.2 mL/10 kg) intra-venously. After 6 weeks' implantation time, the femurs were extracted and placed in a 1× phosphate buffer solution for 2 hours followed by dehydration in a series of ethanol and fixation in 10% formalin for 3 weeks.

Radiograph and Histological Evaluation

A Faxitron LX-60 x-ray system was used to acquire micrographs of the extracted femurs after the PBS wash. Micrographs of each femur were taken at 40 kV with an exposure time 10 s. After fixation, the femurs were embedded in Technovit 7200 and 200-μm sections were cut from the resulting blocks using an Exakt band saw. The sections were then ground and polished using an Exakt grinding system to less than 100 μm and stained with Sanderson's rapid bone stain counterstained with van Gieson. Old allograft bone stained light brown, while new bone stained pink with dark blue osteocytes within the matrix. The polymer was stained dark blue, while cells were stained light blue.

Histomorphometry

A rectangular region approximately 9.5 mm from the plug insertion point across the composite was selected for histomorphometry of the MBP300 and SDBP300 groups. To determine the MBP distribution, a 1.8×3.9 mm rectangle in the unremodeled core was also examined. MetaMorph 7.1 was used to obtain histomorphometry data from the histology micrographs. Differentiation between new bone and cellular infiltration was accomplished using the Smart Brush tool in the Photoshop Elements 7.0 software. The fractions of allograft, cellular infiltration, new bone, and residual polyurethane were measured in the regions of interest. Significant differences between the MBP300 and SDBP300 groups were determined by a Student t-test (p<0.05).

MBP and SDBP Characterization

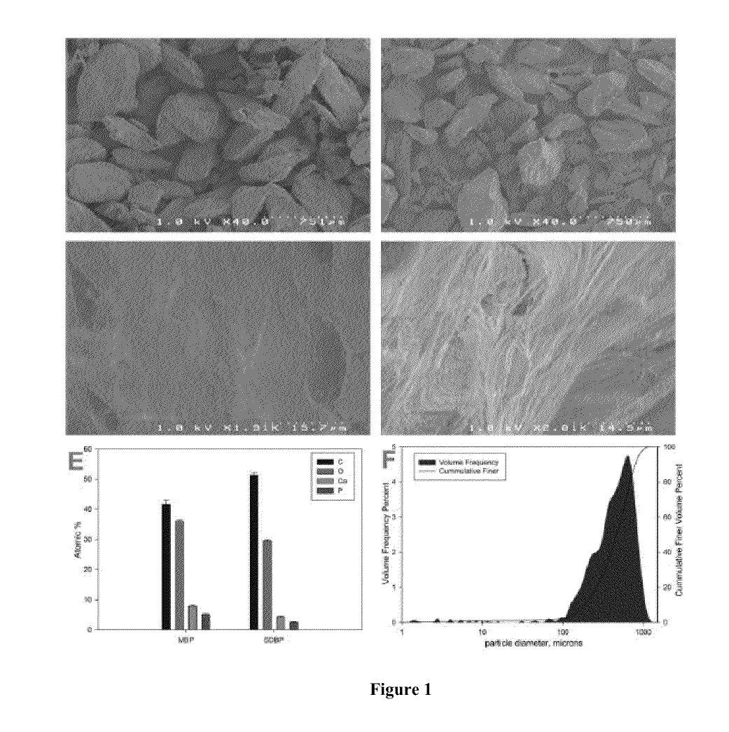

The density of dry MBP was determined by helium pycnometry to be 2.30 g cm⁻³. As evidenced by the low magnification SEM images (FIGS. 1A and 1B), there were insignificant changes in particle size and shape after surface demineralization. Laser light scattering was used to measure the particle size distribution, which was found to be log-normal with a mean value of 481±7 μm (FIG. 1F).

Reactivity of MBP and SDBP Particles

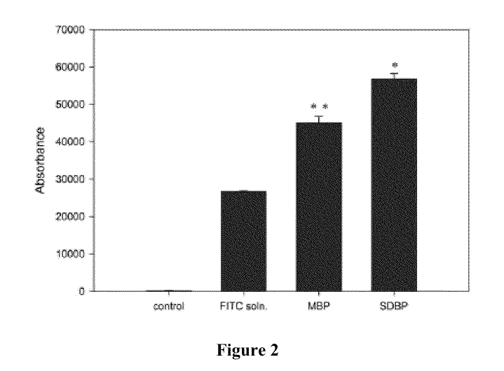

The surfaces of the MBP and SDBP particles were analyzed by XPS to characterize the composition. Surface-demineralization removed a substantial amount of the mineral content at the surface, as evidenced by the significant decrease in Ca and P atomic concentrations and significant increase in C atomic concentration inferred from the XPS spectra (FIG. 1E). The removal of the mineral content was anticipated to increase the reactivity of the surface by exposing a greater number of collagen fibrils at the surface, as shown by the high magnification SEM images in FIGS. 1C and 1D. The higher reactivity of the SDBP particles is demonstrated by the FITC assay (FIG. 2), where active hydrogen (e.g., hydroxyl and amine) groups present in the proteins on the surface of the particles react with the nucleophilic isothiocyanate group (N═C═S) in the FITC molecule. As anticipated, surface demineralization significantly increased the FITC-related absorbance consistent with a significant increase in the number of FITC molecules bound to the surface of SDBP particles compared to MBP. The higher reactivity suggests a higher concentration of active hydrogen molecules on the surface of SDBP, which is anticipated to enhance the mechanical properties of the composite due to the higher degree of interfacial bonding between the allograft filler and reactive two-component PUR binder. However, it is important to note that the fluorescence of the MBP was also higher than that of the FITC-untreated control (MBP in the absence of FITC) and FITC-treated control (tissue culture polystyrene well plate, which is anticipated to have a relatively low reactivity toward FITC).

IR Characterization

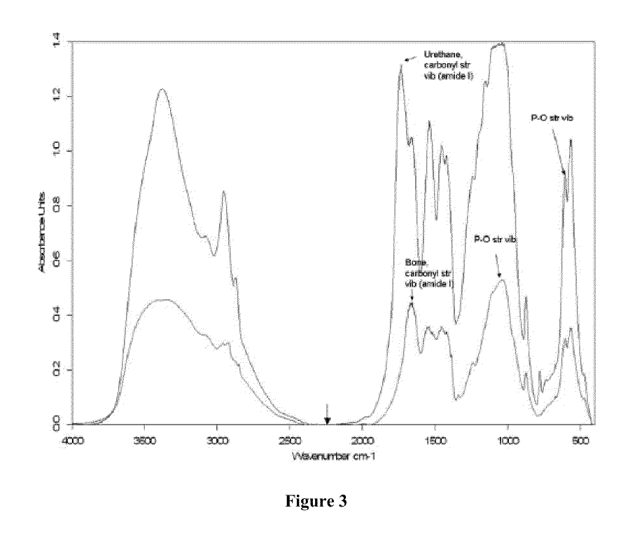

The IR spectrum (FIG. 3) suggests that the PUR phase cured completely, as evidenced by the absence of an NCO peak in the range of 2285-2250 cm^{-1 [46, 60]}. Ester and urethane carbonyl stretching vibrations are observed near 1765 cm^{-1 [40, 46]}. The peaks near 560 and 1030 cm⁻¹ correspond to the phosphate bands in hydroxyapatite that is part of the allograft bone matrix.[61] Thus the IR spectra confirm that the reactive MBP/PUR mixture cured at high conversion to form the expected structure.

Mechanical and Swelling Properties.

The values for the compressive modulus, strength, yield strain, and swelling are listed in Table 2. The modulus and strength values of the composites ranged from 3.05 to 6.01 GPa and 107.8 to 172.4 MPa, respectively. The strain at yield varied from 4.56 to 5.52% while swelling ranged from 2.54 to 2.97%. Composites prepared from the 6C3G1L300 polyester triol exhibited higher strengths and lower strains at yield than the composites based on the 6C3G1L600 triol, presumably due to the higher strength and crosslink density of the polymer binder. The test specimens failed in a diagonal fracture during the compression testing. Surprisingly, surface-demineralization had no effect on the mechanical properties of the composite, as evidenced by the absence of statistically significant differences in swelling or mechanical properties between treatment groups with the same molecular weight polyester triol.

TABLE 2
Mechanical and swelling properties of bone/polymer composites.

| Property | MBP300 | SDBP300 | MBP600 | SDBP600 |
|---|---|---|---|---|
| Compressive modulus, GPa | 6.01 ± 0.34 | 5.52 ± 0.11 | 3.05 ± 0.64 | 3.66 ± 0.39 |
| Compressive strength, MPa | 172.4 ± 4.7 | 166.2 ± 3.8 | 107.8 ± 1.8 | 113.1 ± 3.9 |
| Yield strain, % | 4.56 ± 0.21 | 4.80 ± 0.15 | 5.52 ± 0.57 | 5.77 ± 0.25 |
| Swelling, % | 2.54 ± 0.28 | 2.97 ± 0.27 | 2.89 ± 0.35 | 3.33 ± 0.25 |

Volume Fraction Bone

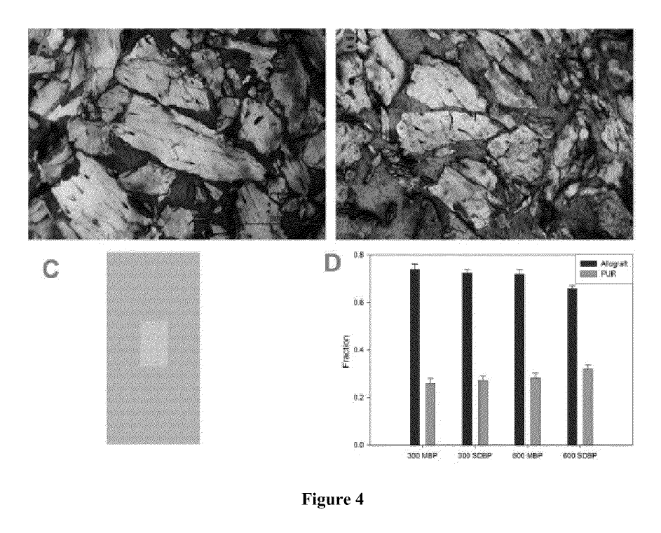

Histological sections near the center of the implants where cells had not yet infiltrated are shown in FIGS. 4A and B. Histomorphometric analysis of the region of the implant shown in FIG. 4C was performed to calculate the volume fractions of bone and polymer for each treatment group. As shown in FIG. 4D, the polymer fraction near the core ranged from 26-32 vol %, while the bone fraction varied from 66-74 vol %. There was a significant difference in bone fraction observed between the 6C3G1L300-MBP and 6C3G1L600-SDBP groups. From the mass balance data, the volume fraction polymer ranged from 32.1-32.4 vol %, while the volume fraction allograft varied from 68.6-68.9 vol %, respectively. Thus the histomorphometric and mass balance data are in agreement that the bone content exceeded the random close-packed (RCP) limit of 64 vol %. Furthermore, the micrographs in FIGS. 4A and B exhibit multiple contacts between adjacent bone particles.

Radiograph Analysis

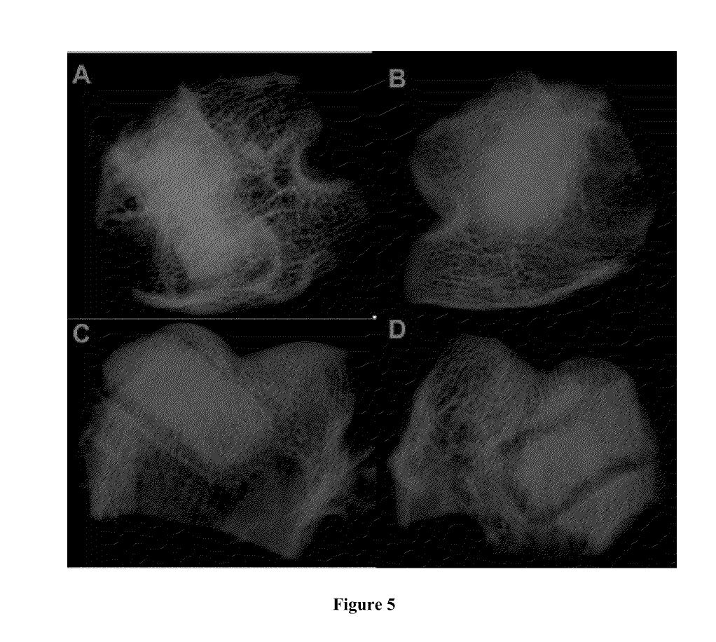

At 6 weeks, the implants were more radiodense than the host trabecular bone allowing the general region of the remaining implant to be evident (FIG. 5). However, regions of host bone immediately surrounding the implant appeared just as radiodense as the implant making the border between the implant and host bone indistinguishable in some areas. Resorption of MBP was observed by the changes in radiodensity within the implant cavity. The radiographs suggest that the composites from the 6C3G1L600-SDBP treatment group resorbed faster than the other groups, as evidenced by the presence of radiolucent zones at the implant margins.

Histological Evaluation

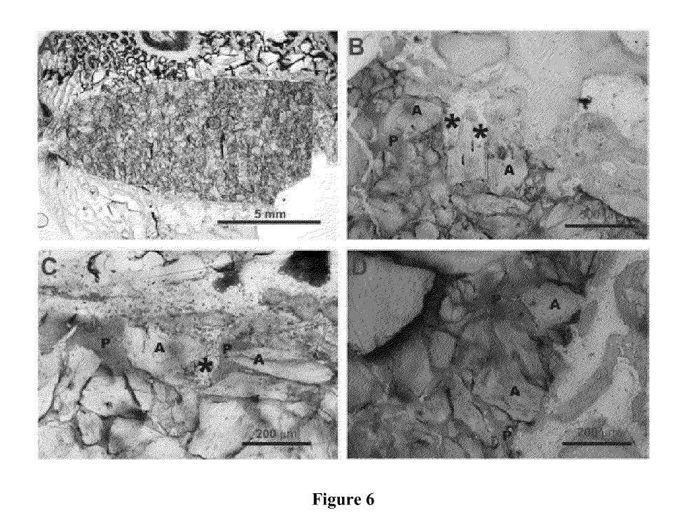

All of the histological micrographs suggest that the PUR/MBP composite plugs were biocompatible, as evidenced by the absence of a significant inflammatory response. Furthermore, the composites did not disrupt the normal wound healing process, as evidenced by the presence of osteoid lining the host bone surrounding the implant. One rabbit that was treated from the 6C3G1L300-SDBP group died at 2 weeks due to causes unrelated to the surgery. As shown in FIG. 6, histological sections processed at this 2 week time point suggest that the MBP/PUR plugs remodeled by the mechanism of creeping substitution.[33, 62] The boundary between the implant and the host bone is well-defined in the low magnification micrograph (FIG. 6A). Growth of new bone in apposition to the surface of the implant followed by the onset of a wall of bone forming around the implant can also be seen (FIG. 6A). The onset of cellular infiltration and resorption of MBP, stained tan/pink, is illustrated in FIGS. 6B-C. Resorption is followed by new bone formation (FIG. 6C). At this early time point, there is minimal degradation of the polymer (blue-green color). Osteoid, stained green, lines the edge of the newly formed bone around the implant in FIG. 6D.

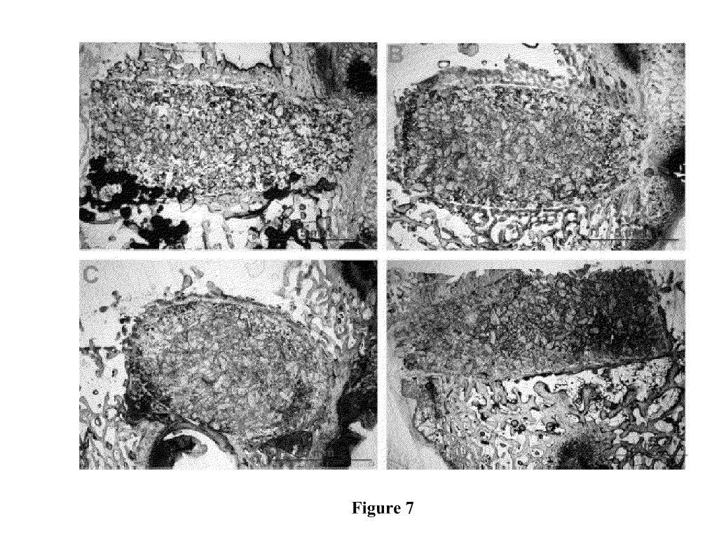

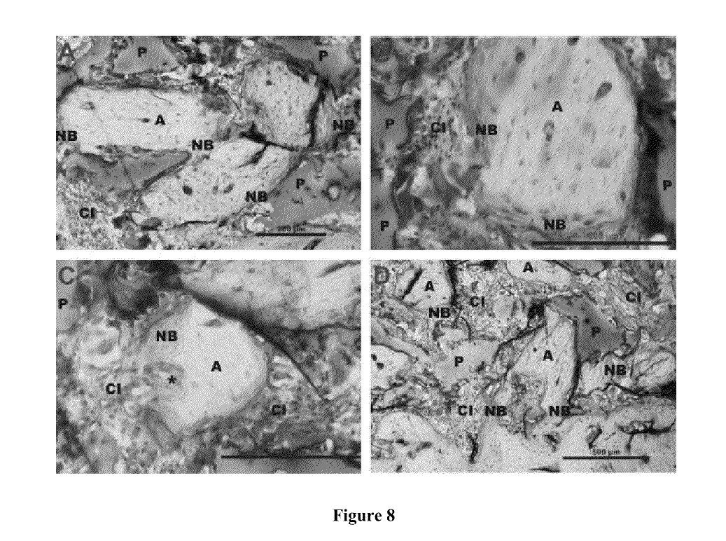

Low magnification micrographs at the 6 week time point (FIG. 7) show differences between treatment groups. In all of the treatment groups, a majority of the resorption, cellular infiltration, and remodeling occurred in the peripheral regions of the implant with little activity occurring in the central core of the implant. The 6C3G1L300-MBP treatment group showed the least cellular infiltration, while the 6C3G1L600-SDBP showed the greatest cellular activity (FIGS. 7A and 7D). There was a significant amount of polymer remaining in all of the treatment groups, especially at the core of the implants. However, composites prepared with the 6C3G1L600 polyol appeared to degrade faster than the materials incorporating the 6C3G1L300 polyester triol (FIG. 7D). The 6C3G1L600-SDBP material supported the most extensive cellular infiltration and polymer degradation. As shown in FIG. 7D, at six weeks cells had infiltrated throughout the entire volume of one end of the implant. Higher magnification micrographs (FIG. 8) show both the resorption of allograft bone particles and new bone formation on their surfaces within the implant cavity. Newly mineralized bone matrix formed on the surface of the allograft particles is evidenced by the more pronounced pink color and the dark blue osteocytes within the matrix. FIG. 8A shows bridging of two allograft particles by new bone. On some allograft particles, both new formation and resorption by osteoclasts appeared to occur simultaneously (FIG. 8C). New bone formation was not limited to the surface of the allograft bone particles, as FIG. 8D shows ingrowth of new bone at the border of the implant. From the images in FIG. 7, remnants of polymer that has not yet resorbed can also be seen. In particular, an island of polymer surrounded by new bone is evident in FIG. 8D. While the continuing presence of the polymer is anticipated to delay new bone formation, especially for the case of bone particles completely embedded in polymer, modest amounts of new bone formed around the polymer remnants. FIG. 8D also shows that the host bone is lined with osteiod, suggesting future ingrowth into the implant cavity.

Histomorphometry

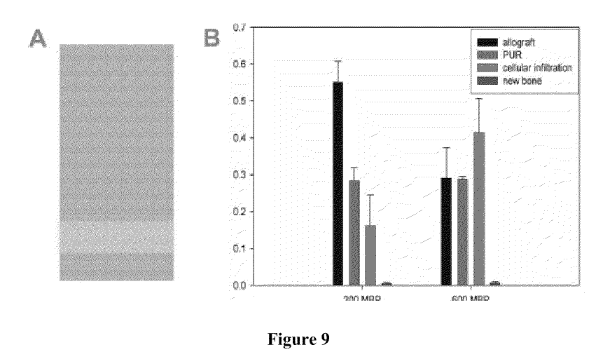

Histomorphometric analysis of the 6C3G1L300-MBP and 6C3G1L600-MBP implants (FIG. 9) was performed to quantify the effects of polyester triol molecular weight on allograft resorption, cellular infiltration, polymer degradation, and new bone formation. After 6 weeks implantation time, the MBP300 implants exhibited 28.3±3.5% residual polymer compared to 29±0.9% for the MBP600 implants, which is not a significant difference. Furthermore, the concentration of polymer at 6 weeks was close to the initial concentration (32.4 vol % from the mass balance), which suggests that the polymer underwent only a modest amount of degradation after 6 weeks. Despite the small differences in polymer resorption at 6 weeks, cellular infiltration and allograft resorption were accelerated in the MBP600 composites, although differences between the two treatment groups were only significant (p<0.06) for allograft resorption. However, although bone resorption and cellular infiltration were higher for the MBP600 composites, the amount of new bone formation was small for both treatment groups (<5%) and the difference between the treatment groups was not significant.

A variety of polymers have been utilized to augment fracture fixation devices and bone replacement materials. While interconnected pores are generally considered necessary to promote bone ingrowth into a polymeric scaffold[3, 4], pre-existing pores significantly reduce the initial load-bearing properties[4] of the device. In the present study, we have fabricated allograft bone/polyurethane composites that have tunable initial mechanical properties comparable to those of host bone. When implanted in plug defects in the femoral condyles of NZW rabbits, the allograft bone component of the composites was resorbed by osteoclasts, thereby creating pores in the composite into which cells infiltrated. Modest polymer degradation and new bone formation were observed. For some of the implants, infiltration of cells deep into the interior was observed after 6 weeks in vivo, which is surprising for solid composites with minimal void space (e.g., <5% porosity).

Several studies have described the preparation of weight-bearing composites incorporating various fillers (such as bioactive glass or hydroxyapatite) for orthopaedic applications. Composites fabricated from synthetic polymers and bioactive glass, which was developed in the early 1970's, have been reported.[63] Young's modulus values as high as 13.6 GPa have been achieved for materials comprising bioglass, urethane dimethacrylate, 2-hydroxylethyl methacrylate, and a photosynthesizing agent.[64] While this value of Young's modulus is close to that of cortical bone, the acrylate polymer component of the bioglass composites was non-degradable. Furthermore, bioactive glass has a slow resorption time, typically greater than 1 year.[1, 65] Thus the combination of a non-degradable polymer and slowly resorbing filler is anticipated to limit the extent of bone ingrowth and remodeling of the composite. Resorbable composite IM rods have been fabricated from hydroxyapatite (HA, 20-30 wt %) and poly(L-lactide) (PLLA) that exhibit bending strength and modulus up to 280 MPa and 7.8 GPa, respectively.[26] Resorption and new bone formation were observed after 5-7 years when HA/PLLA composites were implanted in NZW rabbit femoral plug defects. In a rabbit femoral intramedullary (IM) rod study, bone bridging between HA and host bone was dependant upon the degradation rate of PLLA to allow exposure of HA particles on the surface of the implant.[66] Slowly degrading PLLA implants can take up to 2 years to degrade, leaving behind crystallites that have been reported to induce an inflammatory response. [67] In the metaphyseal region of the rabbit femur, the complete degradation of the PLLA occurred after 4.5 years, while the HA particles were replaced with new bone after 5.5 years.[66] In contrast, the MBP/PUR composites supported rapid bone resorption and cellular infiltration after only 6 weeks in vivo. Since the cells infiltrated the implants through resorption of the nearly continuous MBP phase (as discussed in greater detail below), degradation of the PUR binder was not necessary. The histomorphometry data (FIG. 8) further support the observation that polymer degradation did not precede remodeling, considering that the allograft bone volume fraction decreased from 67.6 vol % to 30-55 vol %, a substantial reduction compared to that observed for the polymer.

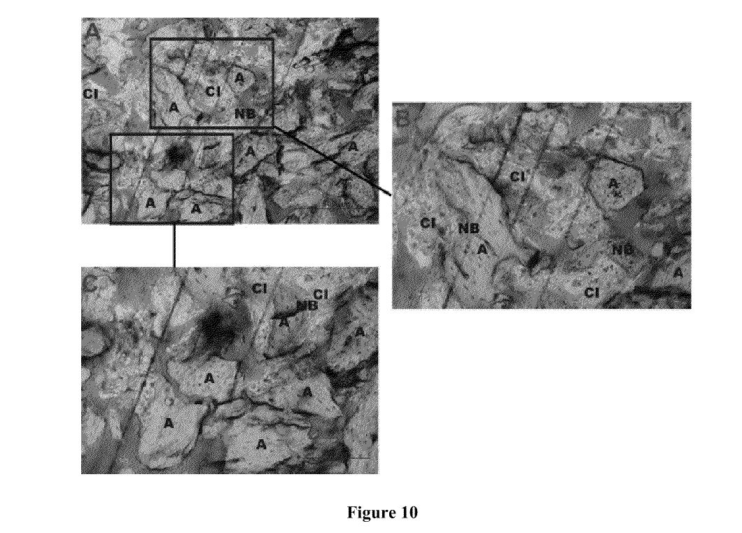

Allograft bone has been a standard of care for the treatment of orthopedic defects because of its osteoconductive properties.[68, 69] However, allograft devices remodel slowly due to the low specific surface area. By combining particulated allograft bone at volume fractions approaching the random close packing limit (64%[70]) with a polymer binder, we reasoned that it would be possible to fabricate composites that undergo more rapid remodeling due to the presence of a nearly continuous allograft bone surface throughout the implant. The extent of bone remodeling in particulated allograft bone/polymer composites has been reported to increase with increasing allograft bone content, with a dramatic increase in both cellular penetration into the implant and new bone formation at 75 wt % (˜61 vol %) bone particles.[36] In the present study, the particulated allograft content was increased to 79 wt % (67.6-67.9 vol % from the mass balance), which slightly exceeded the RCP limit for spheres and approached the limit for acceptable mechanical properties (83 wt %). At the RCP limit, bone particles were in close contact or separated by a thin film, thus presenting a nearly continuous osteoconductive pathway for cells to penetrate the implant by resorbing allograft and migrating into the resulting newly formed pores (FIGS. 10A, B, and C). However, in some cases, non-ideal mixing of the reactive composite paste resulting in polymer-rich regions where the continuous bone phase was partially interrupted (FIG. 10C). While cellular infiltration slowed in the polymer-rich region, cells further infiltrated the implant in an adjacent region where there was closer contact between bone particles (FIG. 10B). Non-ideal mixing is not surprising due to the high viscosity of the reactive two-component PUR binder, especially in the case of the 600 MW groups.

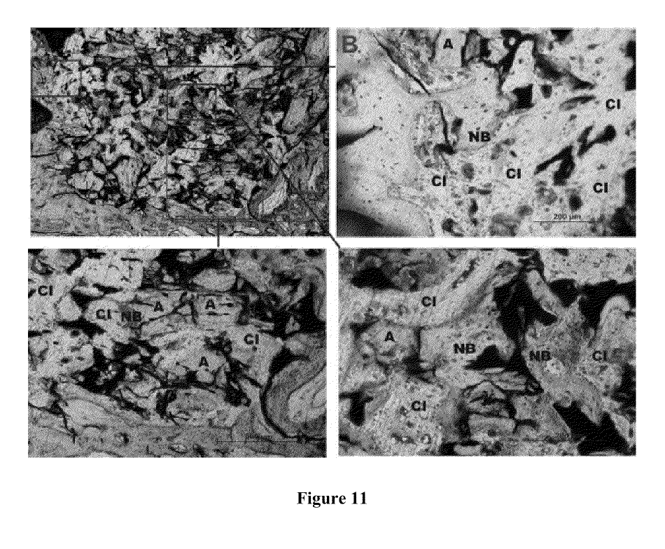

A majority of the composite treatment groups showed increased remodeling activity at the ends of the implant (top and bottom), particularly when the implant was both in direct apposition to the host trabecular bone and exhibited regions enriched in allograft due to non-ideal mixing. FIG. 11 shows the top of a composite from the MBP300 group that underwent both extensive cellular infiltration as well as polymer degradation, and exhibited greater new bone formation. Cellular infiltration, allograft resorption, polymer degradation, and new bone formation were substantially higher in this particular implant compared to other samples in the MBP300 treatment group, presumably due to close contact between an allograft-rich region of the implant and host bone at the base of the implant. With the exception of the implant shown in FIG. 11, composites prepared from the 600 MW groups exhibited faster polymer degradation, cellular infiltration, and allograft resorption due to the lower cross-link density of the PUR networks synthesized from 600 g/mol polyester triols. The dramatically faster rate of remodeling of bone/polymer composites (˜6 wks) relative to the HA/PLLA implants (˜4 yrs) is conjectured to result from either the greater bioactivity of MBP, the presence of a particulated continuous osteoconductive phase, or both. In the MBP/PUR composites, resorption of the bone particles is thus independent of polymer degradation because the particles are already exposed on the surface of the implant, unlike the HA/PLLA composites.

The MBP/PUR implants initially remodeled by creeping substitution, characterized by resorption of allograft followed by new bone formation. [62, 71] However, the rate at which osteoclasts resorbed allograft and cells infiltrated the implant strongly depended on the formulation of the composite (FIG. 7D). Cellular infiltration was highest for the 6C3G1L600-MBP group, where cells had penetrated deep into the interior of the non-porous implant after only 6 weeks. As a result of these processes, an outer ring of demineralized tissue with a modest amount of new bone formation was created around the un-remodeled core. It is conjectured that as the resorption and remodeling proceeds, cells will penetrate further into the core of the implant and new bone will form behind the resorption front, resulting in re-mineralization of the entire implant. Thus the allograft particles function as a biologically active “porogen”, wherein pores are created as the allograft particles are resorbed, followed by cellular migration, matrix deposition, and new bone formation in the newly formed pores. At the short 6 week time point investigated in this study, the amount of new bone formation was modest. Considering that weight-bearing implants must maintain a threshold mechanical strength during the remodeling process, it is desirable that that the demineralized resorption front be as sharp as possible, since a broad resorption front would reduce the mechanical properties of the implant to levels substantially below its initial value. Considering the well-known effects of angio-osteogenic factors, such as rhFGF-2 and rhBMP-2, on enhanced mineralization of porous polymeric scaffolds, it is conjectured that addition of a suitable growth factor would accelerate new bone formation, thereby possibly preserving the weight-bearing mechanical properties of the implant throughout the remodeling process.

Interfacial bonding is well-known to enhance the mechanical properties of composites. The absorbance data in FIG. 2 show that MBP in contact with FITC exhibited a higher absorbance than the negative (MBP+buffer with no FITC) and positive (FITC solution in a tissue culture plastic well plate with no MBP) controls. The higher fluorescent absorbance observed for FITC-treated MBP is conjectured to result from covalent binding of the isothiocyanate (N═C═S) groups in FITC with nucleophiles such as amine and hydroxyl groups present in the proteins in the allograft bone. SDBP treated with FITC exhibited significantly higher absorbance relative to FITC-treated MBP, which is consistent with the XPS data showing that surface demineralization increased the concentration of protein on the surface. These data suggest that the amine and hydroxyl groups on the surface of the allograft particles react with the isocyanate (N═C═O) groups in the LTI to form urea and urethane bonds, respectively, and that surface demineralization would increase the mechanical properties of the composites. Surprisingly, the data in Table 2 show that composites fabricated from SDBP exhibited comparable mechanical properties to those prepared from MBP. Thus while surface-demineralization enhanced the reactivity of the allograft surface, it did not significantly increase the mechanical properties.

The Takayanagi models have been applied to model the mechanical properties of two-phase polymer blends and composites. Assuming the geometry of a circular cross section of the filler is isometric, the Takayanagi models yield the following equations for the compressive modulus E of the composite as a function of the volume fraction and compressive modulus for each phase[49]:

$$\begin{matrix}E = {( \frac{{v}_{1}}{{E}_{1}} + \frac{{v}_{2}}{{E}_{2}} )}^{- 1} & ( 1 ) \\ E = {\sqrt{{v}_{1}} [ \frac{\sqrt{{v}_{1}}}{{E}_{1}} + \frac{1 - \sqrt{{v}_{1}}}{{E}_{2}} ]}^{- 1} + ( 1 - \sqrt{{v}_{1}} ) {E}_{2} & ( 2 ) \\ E = {\sqrt{{v}_{2}} [ \frac{\sqrt{{v}_{2}}}{{E}_{2}} + \frac{1 - \sqrt{{v}_{2}}}{{E}_{1}} ]}^{- 1} + ( 1 - \sqrt{{v}_{2}} ) {E}_{1} & ( 3 ) \\ E = {v}_{1} {E}_{1} + {v}_{2} {E}_{2} & ( 4 )\end{matrix}$$

where v₁ is the volume fraction allograft bone, E₁ is the compressive modulus of the allograft bone particles, v₂ is the volume fraction PUR, and E₂ is the compressive modulus of the PUR component. Eqs (1)-(4) were derived assuming different composite morphologies. Eq (1), which is equivalent to the well-known Reuss model[72], assumes that neither phase is continuous in space, and eq (4), which is equivalent to the well-known Voigt model[72], assumes that both the allograft particles and PUR binder are continuous in space. More physically relevant morphologies intermediate to these upper (Voigt model) and lower (Reuss model) bounds are described by eq (2), which assumes that the PUR binder is continuous, and eq (3), which assumes that the allograft particles are continuous. Values of the composite compressive modulus calculated from each of these conditions are listed in Table 3. A value of 18.6 GPa was used for the modulus of allograft cortical bone.[74] The volume fraction of allograft calculated from the mass balance was ˜68 vol %, which exceeds the spherical random close packing (RCP) limit of 64 vol %. Histomorphometric analysis of the regions near the center of the implant (which were not penetrated by cells) yielded allograft volume fractions ranging from 66-74 vol % (FIG. 4). Qualitative examination of the histological sections showed that the MBP filler was nearly continuous throughout most of the implant, but there were some regions enriched in polymer and depleted in bone particle-particle contacts. Thus, the mass balance and histomorphometric data suggest that the MBP filler was continuous and percolated throughout most of the implants, implying that the compressive modulus of the composites is most accurately predicted by eq (3). Interestingly, the experimental values of the compressive modulus were within 1 GPa of the calculated values assuming a continuous PUR phase, but 3-6 GPa less than those calculated assuming a continuous MBP phase. Considering that surface demineralization enhances allograft reactivity but not composite mechanical properties, insufficient interfacial bonding cannot explain the lower experimental values of the compressive modulus relative to the Takayanagi model predictions. Closer examination of the histological sections near the core (FIG. 4) revealed that not all of the particle-particle interactions were point contacts, but rather extensive areas of contact where there was minimal polymeric binder present between the allograft particles, thereby creating defects along which cracks could propagate. However, it is conjectured that these defects also accelerated allograft resorption by increasing the area available for cellular infiltration. Thus biomechanics and remodeling are inter-related, such that the mechanical properties are reduced as the RCP limit is approached, but the processes of resorption and cellular infiltration are accelerated.

TABLE 3
Takayanagi model calculations for compressive modulus of
bone/polymer composites. All composites incorporated 79 wt % allograft bone particles. E_{C}
denotes calculated compressive modulus calculated from the Takayanagi models.

| Property | MBP300 | SDBP300 | MBP600 | SDBP600 |
|---|---|---|---|---|
| Bone density, g cm⁻³ | 2.30 | 2.30 | 2.30 | 2.30 |
| PUR density, g cm⁻³ | 1.274 ± 0.005 | 1.274 ± 0.005 | 1.290 ± 0.003 | 1.290 ± 0.003 |
| Bone modulus, GPa | 18.6 | 18.6 | 18.6 | 18.6 |
| PUR modulus, GPa | 1.427 ± 0.039 | 1.427 ± 0.039 | 0.988 ± 0.055 | 0.988 ± 0.055 |
| Volume fraction bone, % | 67.6% | 67.6% | 67.9% | 67.9% |
| Volume fraction polymer, % | 32.4% | 32.4% | 32.1% | 32.1% |
| E_{C}, both phases discont., GPa | 3.79 | 3.79 | 2.76 | 2.76 |
| E_{C}, PUR continuous, GPa | 5.12 | 5.12 | 3.87 | 3.87 |
| E_{C}, MBP continuous, GPa | 9.36 | 9.36 | 9.00 | 9.00 |
| E_{C}, PUR and MBP continuous, | 13.0 | 13.0 | 12.9 | 12.9 |
| GPa | | | | |
| Measured modulus, GPa | 6.01 ± 0.34 | 5.52 ± 0.11 | 3.05 ± 0.6 | 3.66 ± 0.39 |

Non-porous MBP/PUR composites are a high strength, osteoconductive biomaterial suitable with initial mechanical properties suitable for weight-bearing applications. The mechanical properties and cellular infiltration rate can be tuned for specific applications by manipulating the molecular weight of the polyester polyol used during synthesis. Cellular infiltration and new bone formation were observed in the interior of the implant at 6 weeks, which is surprising for composites with such low porosity (<5%). Osteoclast-mediated resorption of the allograft particles created pores into which cells migrated, followed by deposition of new collagen matrix and bone formation. Due to the time lag between resorption and re-mineralization, a de-mineralized resorption front was observed at 6 weeks, which is anticipated to reduce the mechanical properties as the implant remodels. Although further time points are needed to investigate the full resorption and re-mineralization profile, the findings from this study suggest that MBP/PUR composites may have potential application as biologically active weight-bearing devices for bone tissue engineering.

### Example 2

### Materials

Methyl 2,6-diisocyanatohexane (lysine methyl ester diisocyanate, LDI) was purchased from Kyowa Hakko USA (New York). Coscat 83, an organobismuth catalyst, was provided by ChasChem, Inc. (Rutherford, N.J.). Poly(ε-caprolactone) triol (300 Da) (PCL 300) was purchased from Sigma Aldrich (St. Louis, Mo.). Trypsin was purchased from Fisher Scientific.

Quasi-Prepolymer Synthesis.

An appropriate amount of Coscat 83 was added to a 50 mL three neck flask, followed by the addition of LDI. The Coscat 83 and LDI was mixed using a magnetic stir bar and allowed to reach 90° C. using a heating mantel. A nitrogen line was connected to one neck of the flask, and a condenser was connected another neck of the flask. Once the mixture reached the target temperature of 90° C., PCL 300 was added to drop wise to the flask via a syringe through the center neck of the flask. The reaction was allowed to proceed for 3 hours, and the resulting quasi-prepolymer (QP) was poured into a storage jar which was purged with additional nitrogen. The QP was stored in a Fisher Isotemp refrigerator at 4° C.

Synthesis of MBP/Polyurethane Composites.

Additional Coscat 83 was added to a 10 mL mixing cup to yield a final concentration of 1500 ppm, followed by polyester triol. Both PCL 300 and synthesized polyester triol co-polymers were used as the soft segment of the MBP/PEUR composites. The method for polyester triol co-polymer synthesis was adapted from previously published techniques. MBP was added to the cup and its contents were mixed for 30 s at 3300 rpm using a Hauschild SpeedMixer. After mixing, quasi-prepolymer was added to the cup and mixed for an additional 15 s at 3300 rpm.

Composites were also prepared using a one-shot process as described in Example 1.

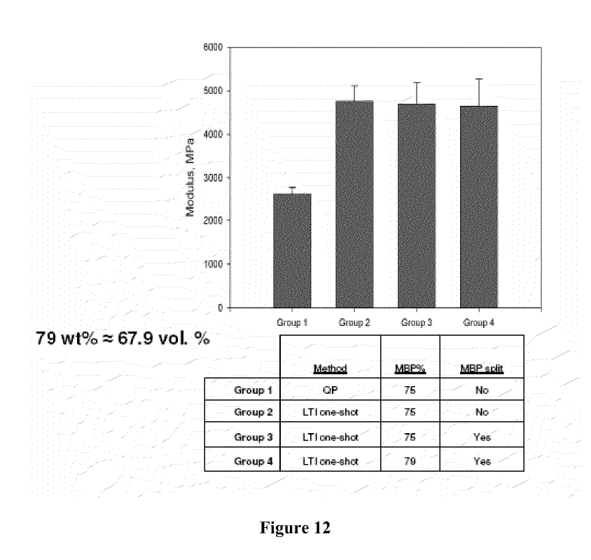

There are two procedures for making the composites in FIG. 12: (a) LDI quasi-prepolymer (QP) and (b) one-shot. Group 1 was made using QP process. Group 2 was made using one-shot process. Groups 3 and 4 are the same as Group 2 except for the bone fraction and MBP split, which means in group 4 the MBP was split between the LTI and the polyol components instead of adding it only to polyol component. As the data show there is no effect on modulus. To convert wt % of bone fraction to vol % use the following formula:

phi=vol fraction={x(B)/rho(B)}/{x(B)/rho(B)+x(P)/rho(P)},

where x=weight fraction, B denotes bone, P denotes polymer, rho(B)=2.1 g/cm̂3 is bone density and rho(P)=1.2 g/cm̂3 is polymer density.

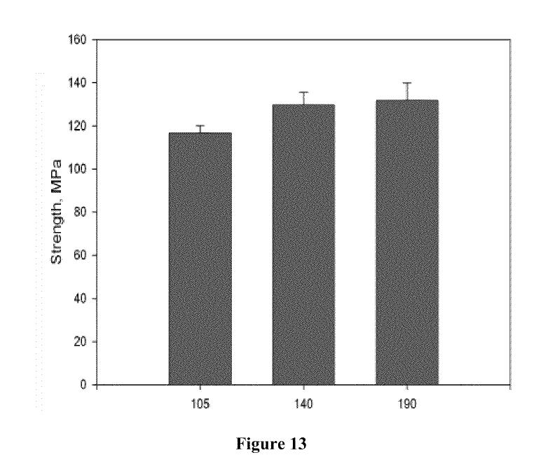

FIG. 13 shows LTI one-shot approach shits optimal index (on the x-axis).

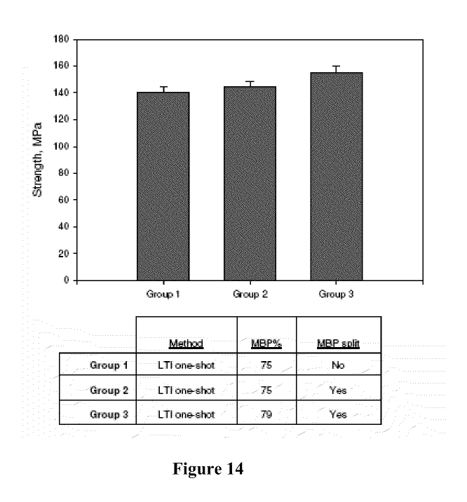

FIG. 14 shows no effect of splitting the bone between the LTI or polyol. In addition, more bone in the composites resulted in higher compressive strength.

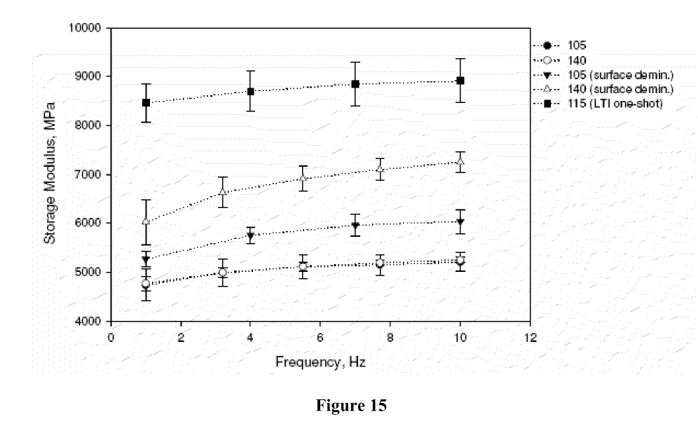

These are dynamic mechanical analysis data in FIG. 15 showing index effects. samples were dry. four of the samples were QP materials showing index effects. The one labeled 115 (LTI one-shot) was prepared using the one-shot process. Data shows that 115 index LTI one-shot gives higher bending modulus (measured in 3 point bending mode) than any of the QP composites at index 105-140. These are DMA data, so storage modulus is plotted as a function of frequency.

All references, such as patents, patent applications, and publications, referred to above are incorporated by reference in their entirety.

Other embodiments are within the scope of the following claims.

1. A composite comprising:
a plurality of bone particles, and polyurethanes with which the bone particles have been combined,
wherein the composite has wet compressive strength more than 50 MPa.

2. A composite comprising:
a plurality of bone particles, and polyurethanes with which the bone particles have been combined,
wherein the composite has wet compressive modulus more than 1 GPa.

3. A method of preparing a weigh-bearing composite comprising steps of:
preparing a composition that comprises a polyol;
contacting the composition with a polyisocyanate; and
adding at least 45 vol % particles.

4. The method of claim 3, wherein the composites are prepared in a one-shot process.

5. The method of claim 3, wherein the composites are prepared by compression molding.

6.-10. (canceled)

11. The method of claim 3, wherein the bone particles are nondemineralized, superficially, partially or fully demineralized.

12. The method of claim 3, wherein the bone particles are defatted.

13. The method of claim 3, wherein a mean average particle size of the bone particles is in a range of about 100 to about 1000 microns, about 200 to about 800 microns, or about 300 to about 600 microns.

14. The method of claim 3, wherein the bone particles are fibers.

15. The method of claim 3, wherein the bone particles are elongated particles.

16. The method of claim 3, further comprising a step of adding an inorganic material.

17. The method of claim 16, wherein the inorganic material is selected from the group consisting of aragonite, dahlite, calcite, amorphous calcium carbonate, vaterite, weddellite, whewellite, struvite, urate, ferrihydrite, francolite, monohydrocalcite, magnetite, goethite, dentin, calcium carbonate, calcium sulfate, calcium phosphosilicate, sodium phosphate, calcium aluminate, calcium phosphate, hydroxyapatite, α-tricalcium phosphate, dicalcium phosphate, β-tricalcium phosphate, tetracalcium phosphate, amorphous calcium phosphate, octacalcium phosphate (OCP), BIOGLASS™, fluoroapatite, chloroapatite, magnesium-substituted tricalcium phosphate, carbonate hydroxyapatite, and combinations and derivatives thereof.

18. The method of claim 3, wherein the polyol comprise a polymer selected from the group consisting of poly(caprolactones), poly(lactide), poly(glycolide), polyglyconate, poly(arylates), poly(anhydrides), poly(hydroxy acids), polyesters, poly(ortho esters), poly(alkylene oxides), polycarbonates, poly(propylene fumarates), poly(propylene glycol-co fumaric acid), polyamides, polyesters, polyethers, polyureas, polyamines, polyamino acids, polyacetals, poly(orthoesters), poly(pyrolic acid), poly(glaxanone), poly(phosphazenes), poly(organophosphazene), polylactides, polyglycolides, poly(dioxanones), polyhydroxybutyrate, polyhydroxyvalyrate, polyhydroxybutyrate/valerate copolymers, poly(vinyl pyrrolidone), polycyanoacrylates, polyurethanes, polysaccharides, KRYPTONITE, and combinations thereof.

19. The method of claim 3, wherein the polyol comprise poly(caprolactone), poly(lactide), poly(glycolide), and/or combinations thereof.

20. The method of claim 3, wherein the polyisocyanate comprise LTI.

21. (canceled)

22. The method of claim 3, wherein the composition further comprises a catalyst.

23. The method of claim 22, wherein the catalyst comprises a tertiary amine.

24. The method of claim 3, wherein the composite further comprises a bioactive agent to be delivered.

25. The method of claim 24, wherein the bioactive agent is selected from the group consisting of antiviral agent, antimicrobial agent, antibiotic agent, amino acid, peptide, protein, glycoprotein, lipoprotein, antibody, steroidal compound, antibiotic, antimycotic, cytokine, vitamin, carbohydrate, lipid, extracellular matrix, extracellular matrix component, chemotherapeutic agent, cytotoxic agent, growth factor, anti-rejection agent, analgesic, anti-inflammatory agent, viral vector, protein synthesis co-factor, hormone, endocrine tissue, synthesizer, enzyme, polymer-cell scaffolding agent with parenchymal cells, angiogenic drug, collagen lattice, antigenic agent, cytoskeletal agent, mesenchymal stem cells, bone digester, antitumor agent, cellular attractant, fibronectin, growth hormone cellular attachment agent, immunosuppressant, nucleic acid, surface active agent, and penetraction enhancer.

26. (canceled)

27. A method of preparing a composite comprising steps of:
providing the composite comprising particles and polymer components in a flowable or moldable state that is characterized by having less than 70 vol % particles and;
allowing the composite to transition to a hardened state that is characterized by having wet compressive strength of more than 50 MPa.

28. (canceled)
